(19) United States
(12) Patent Application Publication
(10) Pub. No.: US 2006/0241150 A1
(43) Pub. Date: Oct. 26, 2006

(54) P38 kinase inhibitor compositions and methods of using the same

(76) Inventors: David B Weiner, Merion, PA (US); Karuppiah Muthumani, Cherry Hill, NJ (US)

Correspondence Address: COZEN O'CONNOR, P.C., 1900 MARKET STREET, PHILADELPHIA, PA, 19103-3508, US

(21) Appl. No.: 10/559,227

(22) PCT Filed: Jun. 4, 2004
(86) PCT No.: PCT/US04/17696

§ 371 Date: May 24, 2006

(30) Foreign Application Priority Data
Jun. 6, 2003 (US) 60476397

(51) Int. Cl. A61K 31/4439; A61K 31/551; A61K 31/522; A61K 31/513; C12Q 1/70

(52) U.S. Cl. 514/338; 514/220; 514/263.32; 514/269; 435/5

(57) ABSTRACT

Methods of treating an individual who has been identified as having been infected with HIV are disclosed. Methods of treating an individual who is suspected of having been exposed to HIV are disclosed. Some methods disclosed comprise the step of administering to the individual an amount of a p38 inhibitor effective to inhibit FasL expression. Some methods disclosed comprise the step of administering to the individual an amount of a p38 inhibitor effective to inhibit HIV replication. Some methods disclosed comprise the step of administering to the individual an amount of a p38 inhibitor effective to inhibit HIV replication without inhibiting T cell activation. Some methods disclosed comprise administering to the individual one or more other anti-HIV compounds in combination with a p38 inhibitor. Methods of identifying compounds that inhibit Nef mediated upregulation of FasL expression are disclosed. Methods of identifying compounds that inhibit the p38 pathway are disclosed.

### FIELD OF THE INVENTION

The present invention relates to compositions comprising p38 kinase inhibitors as anti-HIV compositions and the use of such compositions to treat and prevent HIV infection.

### BACKGROUND OF THE INVENTION

Human Immunodeficiency Virus type 1 (HIV-1) is the infectious agent responsible for AIDS, currently one of the world's foremost health problems. In the United States alone there are over 40,000 new HIV infections per year. Globally, countries with established market economies have over 1.2 million current infections, while in developing nations close to 45 million infections are estimated and over 20 million people have already died from AIDS. Recent attempts to develop vaccines for HIV have met with significant frustration. There is still no immunogen that can induce broadly neutralizing antibody responses. Much energy has been focused on the development of cellular methods of inducing protection in non-human primate models of HIV. However, problems of viral escape and unexpected haplotype based protective responses in non-human primate studies provide challenges to vaccine design. As no vaccine will likely be commercially available for at least several years there appears to be a great need for additional novel therapeutic agents for this uncontrolled infection.

Accordingly, the development of new strategies to treat HIV infection is a high priority. Currently just over 54 drugs have FDA approval for treating HIV and AIDS-related ailments. However, historically, the vast majority of the orally deliverable compounds that target HIV genes, target two enzymes within the viral pol gene complex. It is frustrating that, in all cases, these approaches appear to be associated with viral escape. Recent progress in the development of fusion inhibitors has broadened the HIV targets to include envelope gene products as well. Significant toxicities associated with long-term use of any of these agents are a concern. Of further concern is that, it appears that even heavily treated individuals are not able to clear the underlying HIV viral infection, or prevent viral replication. Thus, it is believed that over time the vast majority of treated individuals will require additional therapies. In theory, developing new therapies, that target host cellular pathways that are utilized by the virus, could impose additional important and valuable obstacles for the virus. A particular benefit would be if such a host pathway interfered with a currently untargeted pathogenic gene product of HIV-1.

The human immunodeficiency virus type 1 genome comprises two structural gene segments (gag and env) and enzymatic gene complex pol similar to prototypic members of the retroviral family. HIV-1 also encodes a number of regulatory and accessory genes that have diverse roles in the virus life cycle and are implicated in viral pathogenesis. In this regard, disregulated apoptosis is considered a major pathogenesis event leading to severe CD4⁺ lymphopenia during the human immunodeficiency virus type 1 (HIV-1) infection. Apoptosis is a mode of cell death that occurs under normal physiological conditions in which the cell is an active participant in its own demise. Cells undergoing apoptosis show characteristic morphological and biochemical features. HIV induced apoptosis has been reported to both involve and not involve the Fas/FasL apoptotic pathway. CD95 (APO-1/Fas) is a member of the TNF receptor superfamily expressed on various tissues, whereas expression of its ligand (CD95L), a type II transmembrane protein of the TNF family is more restricted to a few cell types, such as T-cells, macrophages and cells of the testis. FasL is not present in resting T cells but activated T cells may undergo apoptosis using the CD95/CD95L pathway. Exactly how and which HIV gene products activate apoptosis through the Fas/FasL pathway is an important topic of investigation. The viral gene products vpr, nef, tat and env have all been reported to drive apoptosis to varying extents in several model systems of HIV infection and all four of these gene products have been implicated to varying degrees in bystander cell killing. While the env gene product is important in viral entry and tropism, the tat, vpr and nef gene products are all also implicated as having direct relevance to viral replication. Tat is absolutely necessary for high levels of viral transcription, while Nef has been shown to be critical for induction of high viral loads and viral pathogenesis in SIV model systems.

There remains a need for additional drugs and methods for treating HIV infection.

### SUMMARY OF THE INVENTION

The present invention relates to methods of treating an individual who has been identified as having been infected with HIV. The methods comprise the step of administering to the individual an amount of a p38 inhibitor effective to inhibit FasL expression.

The present invention further relates to methods of treating an individual who is suspected of having been exposed to HIV. The methods comprise the step of administering to the individual an amount of a p38 inhibitor effective to inhibitFasL expression.

The present invention further relates to methods of treating an individual who has been identified as having been infected with HIV. The methods comprise the step of administering to the individual an amount of a p38 inhibitor effective to inhibit HIV replication without inhibiting T cell activation.

The present invention further relates to methods of treating an individual who is suspected of having been exposed to HIV. The methods comprise the step of administering to the individual an amount of a p38 inhibitor effective to inhibit HIV replication without inhibiting T cell activation.

The present invention provides methods of inhibiting FasL expression in an HIV infected cell comprising the step of delivering to the HIV infected cell an amount of a p38 inhibitor sufficient to inhibit FasL expression in said cell.

The present invention additionally provides methods of inhibiting HIV replication in an HIV infected cell comprising the step of delivering to the HIV infected cell an amount of a p38 inhibitor that does not inhibit T cell activation sufficient to inhibit HIV replication.

The present invention furiher relates to methods of identifyg compounds that inhibit Nef mediated upregulation of FasL expression. The methods comprising performing a test assay that comprises the steps of contacting a cell that expresses Nef, which upregulates FasL expression in the cell, with a test compound. The level of FasL expression is measured and compared to the level in the absence of the test compound.

The present invention further relates to methods of identifying compounds that have anti-HIV activity. The methods comprising performing a test assay that comprises the steps of contacting a cell that expresses Nef, which upregulates FasL expression in the cell, with a test compound. The level of FasL expression is measured and compared to the level in the absence of the test compound.

The present invention further relates to methods of identifyig compounds that inhibit the p38 pathway. The methods comprising performing a test assay that comprises the steps of contacting a cell that expresses Nef, which upregulates FasL expression in the cell, with a test compound. The level of FasL expression is measured and compared to the level in the absence of the test compound.

The present invention further relates to methods of identifying compounds that inhibit the JNK pathway. The methods comprising performing a test assay that comprises the steps of contacting a cell that expresses Nef, which upregulates FasL expression in the cell, with a test compound. The level of FasL expression is measured and compared to the level in the absence of the test compound.

### BRIEF DESCRIPTION OF THE FIGURES

FIGS. 1A-1D present data showing that HIV-1 induces apoptosis in human PBMCs, and that such apoptosis is inhibited by p38 MAP kinase inhibitors. FACS analysis of Annexin V-FITC stained cells were done on cells either infected with HIV-1 89.6 (FIG. 1A), or pNL4-3 having different clade specific primary viral isolates as indicated (FIG. 1B or 1C). Cells were either uninfected mock, infected with HIV-1 virus, infected with virus and treated with 1 μM of SB203580, or infected with virus and treated with 1 μM of Cpd4 inhibitor. Cells were collected 4 days post infection and specific inhibitor treatment then stained with Annexin V-FITC as described in Materials and Methods. Numbers appearing in the panel indicates the percent Annexin V positive. Significant inhibition of apoptosis by p38 inhibitors is apparent. FIG. 1D present data showing that selective inhibitors of p38 MAP kinase block Caspase 3 activity. Cell lysates were prepared from the infected cells. 100 μg of protein from each cell lysate was used for the colorimetric protease assay as described in the Materials and Methods. Each column represents the mean standard deviation of results from three sarmples in three independent experiments. Groups tested Mock (uninfected), virus infected, virus infected+Cpd4, and virus infected+SB203580.

FIGS. 2A-2C present data showing HIV infection upregulates FasL expression and that FasL expression is inhibited by p38 inhibitors FIG. 2A shows data from flow cytometry of FasL expression in mock or HIV-infected cells (89.6 or pNL4-3 virus) or PBMCs and or Jurkat T cells. FIG. 2B shows data of FasL expression in mock-infected or HIV-infected Jurkat T cells in the presence of 1 μM of Cpd4. FIG. 2C shows data of FasL expression in mock-infected or HIV-infected PBMCs in the presence of 1 μM of Cpd4. Cells were harvested 2 days post infection and stained with anti-FasL antibody (NOK-1) PE. Data is representative of 3 different experiments with three different donors studied.

FIGS. 3A and 3B present data from comparisons of the FasL expression induction by individual HIV-1 genes. Jurkat T cells were electroporated with 20 μg of pCDNA 3.1 (mock), pCDNA-Env, pCDNA-Tat, pCDNA-Vpr or pCDNA-Nef plasmids. Forty-eight hours after transfection, the surface levels of FasL expression were determined by flow cytometry after staining with a FasL-specific antibody (NOK-1). FIG. 3A shows that Nef was a dramatic induces of FasL expression on T cells. FIG. 3B shows Nef induction of FasL is highly suppressed by Cpd4. Filled histograms represent FasL expression levels and dotted lines represent the IgG isotype control.

FIGS. 4A-4C present data relevant to Nef constructs, expression and effects. FIG. 4A present schematic representations of the HIV-1 pNL4-3 proviral expression constructs. pNL4-3 wt or the pNL4-3 frameshift (5′ Nef). FIG. 4B show data from Nef expression analysis of proviral constructs. Cell lysates from 293T cells transfected with the HIV-1 proviral constructs pNL4-3 wt or the pNL4-3 Nef(−), or from cells transfected with the Nef-encoding plasmid pCNef or from cells infected with wild type 89.6 virus were separated by 12% SDS-PAGE and then transferred to nitrocellulose filters. Lysates were probed with an HIV-1 Nef specific antiserum. FIG. 4C shows data measuring viral production after infections by measuring p24 levels in the culture supernatants of pNL4-3 Wt or pNL4-3/delta Nef. Data presented reflects the measurement 96 hrs post infection.

FIG. 5 shows data from experiments studying induction of FasL by Nef positive viruses in human PBMCs. Cells (1×10⁶ cells) were infected with pNL4-3 Wt or pNL4-3 delta Nef virions and infected cells were then treated with 1 μM Cpd4 compound or control as indicated. Two days post infection and treatment an equal number of cells (1×10⁶) was studied for p24 expression and their expression of p24^{gag}-FITC or CD95L-APC was plotted. These experiments were repeated three times and similar results were obtained. Nef positive virus induction of FasL is completely inhibited by Cpd4 at a 1 μM concentration.

FIGS. 6A and 6B show data from various experiments described herein and below. FIG. 6A shows data from experiments in which CD14⁺ macrophages were prepared from naïve patients and stimulated and then infected with 89.6 virus (100 TCID₅₀/1×10⁶ cells/ml) washed and incubated for 3 days and then mixed with autologous CD8⁺ T cells. 12 hrs later cells were stained with Annexin V and apoptosis induction in the CD8⁺ population was studied (panel iv). CD8⁺ T cell apoptosis was induced as detected by flowcytometry. FIG. 6B shows data from experiments in which uninfected (i) and HIV infected (ii) autologous macrophages were prepared as in FIG. 6A and were stimulated with polystyrene latex beads and incubated with purified uninfected CD8⁺ T cells in the presence of neutralizing anti-FasL antibody, or with 1 μM Cpd4 and incubated overnight. Cells were then harvested and stained for Annexin V as described in Materials and Methods. The values of each quadrangle represent the Annexin V expression in %. The experiment was repeated twice with similar results.

FIGS. 7A-7D show data comparing effects by Nef and Nef+Cpd4 HIV-1 Nef activates transcription factors which can be inhibited by Cpd4. Jurkat T-cells were transfected with AP-1 (FIG. 7A) or NF-κB (FIG. 7B) dependent reporter plasmid (1 μg) and pNef plasmid (1 μg) and treated with or without Cpd4 (1 μM) as indicated. Cells were lysed after 24 hours and assayed for luciferase activity. Results were normalized to control transfected β-gal levels. The experiment was conducted three times with similar results. Biochemistry of Nef Activation is shown in FIG. 7C. Cells were treated as described above and then analyzed for p38 activation. Total protein was extracted as described in Materials and Methods and resolved on a 12% SDS-PAGE gel normalized for protein quantity. Following gel preparation the gels were transferred to filters and immunobloted with an anti-p38 or phospho p38 antibody. Nef induces phosphorylation and activation of p38 and this activation can be blocked by p38 inhibitors. Nuclear extracts of similarly treated cells were analyzed for c-Jun activation. As with the p38 pathway, Nef induced strong activation of c-Jun, which can be blocked by Cpd4 (FIG. 7D).

FIG. 8 shows the structures of Cpd4 and SB203580.

### DESCRIPTION OF PREFERRED EMBODIMENTS

Definitions

As used herein, the terms “an amount effective to inhibit Nef mediated upregulation of FasL expression in HIV infected cells” and “an amount effective to inhibit FasL expression in HIV infected cells” refer to the amount of a p38 inhibitor administered to an individual that results in the HIV-infected T cells of the individual expressing less FasL. To determine such amounts, the amount of FasL present on HIV infected T cells can be counted prior to treatment with a p38 inhibitor compound and then subsequent to treatment. FasL expression can be quantified by any number of routine methodologies including FLOW, cytometry using blood samples taken from patients. The down modulation of FasL expression contributes to a reduction in the severity of the infection or symptoms therefrom.

As used herein, the term “an amount effective to inhibit HIV replication” refers to the amount of p38 inhibitor administered to an individual that results in a reduced level of HIV replication and thus a reduced amount of detectable virus in the individual, i.e a reduction in viral titer or viral load. To determine an amount effective to inhibit HIV replication, the individual's viral load can be determined prior to treatment with a p38 inhibitor compound and then subsequent to treatment. The level of HIV replication can be quantified by any number of routine methodologies including, for example: quantifyig the actual number of viral particles in a sample prior to and subsequent to p38 inhibitor administration, quantifying the level of HIV antigen, such as p24, present in a sample prior to and subsequent to p38 inhibitor administration, and quantifyig the level of reverse transcriptase or HIV protease activity or titer in a sample prior to and subsequent to p38 inhibitor administration. The inhibition of HIV replication contributes to a reduction in the severity of the infection or symptoms therefrom.

As used herein, the term “without inhibition of T cell activation” refers to an absence of reduction in the ability of T cells to be activated. Accordingly the term “an amount effective to inhibit HIV replication without inhibition of T cell activation” refers to an amount of p38 inhibit or that inhibits HIV replication but does not inhibit T cell activation. To determine an absence of inhibition of T cell activation, the ability of T cells to become activated in a patient is determined prior to and subsequent to administration of the p38 inhibitor. Standard and routine methods may be used to determine levels of T cell activation such as IL-2 production by PBMC contacted with SEB superantigen. The absence of inhibition of T cell activation provides the patient, who may be immunocompromised, the advantage of not inhibiting their immune system and thus making the patient better able to fight the HIV infection as well as any other infectious agents such as opportunistic infections that ARC and AIDS patients can be particularly susceptible to.

As noted, level FasL expression, level of HIV replication and level of T cell activation can be determined routinely using well known techniques. Several techniques are disclosed herein or Wadsworth SA, et al 1999 Pharmacol Exp Ther. 291(2):680-687, which has been incorporated herein by reference. Generally, the threshold for inhibition of FasL expression or HIV replication would be that p38 inhibitor treated would be at least 10% less than untreated, preferably at least 25% less than untreated. Generally, the threshold for the absence of inhibition of T cell would be that T cell activation levels in p38 inhibitor treated would be within 10% of that T cell activation levels in untreated, preferably within 20% of that T cell activation levels in untreated.

The p38 kinase, also known as p38 MAP kinase (the two terms being used herein interchangeably), is a kinase that is normally activated in response to stress.

As used herein, the terms “p38 inhibitor,” “p38 kinase inhibitor,” and “p38 MAP kinase inhibitor” are used interchangeably and meant to refer to a compound that is capable of inhibiting p38 MAP kinase activity. The compound can be a small molecule, large molecule, peptide, oligonucleotide, and the like. The determination of whether or not a compound is a p38 kinase inhibitor is within the skill of one of ordinary skill in the art. An example of how one would determine if a compound is a p38 kinase inhibitor would be to isolate the p38 kinase protein. The protein can be isolated from cells where the p38 kinase is naturally expressed or where it has been overexpressed by means of transfection of an oligonucleotide or infection with a virus that directs the expression of the p38 MAP kinase protein. Additionally, p38 can also be expressed recombinantly. Upon isolating the protein a person of ordinary skill in the art can measure the activity of the kinase in the presence or absence of a potential p38 kinase inhibitor. If the kinase activity is less in the presence than in the absence of an alleged inhibitor, that inhibitor is a p38 kinase inhibitor.

As used herein, the term “an individual suspected of having been exposed to HIV”refers to an individual who has not been diagnosed as being HIV positive but who could possibly have been exposed to HIV due to a recent high risk activity or activity that likely put them in contact with HIV. For example, an individual suspected of having been exposed to HIV refers to an individual that has been stuck with a needle that has been in contact with either a sample that contains HIV or HIV infected individual. Examples of samples include, without limitation, laboratory or research samples or samples of blood, semen, bodily secretions, and the like from patients. Other individuals suspected of being exposed to HIV include individuals that have received blood transfusions with blood of unknown quality. The blood that is being transfused may have not been tested or the test results indicating that the blood does not contain HIV are not reliable or are doubted. In some embodiments, an individual suspected of being infected with HIV includes individuals who have had, sexual intercourse, unprotected sexual intercourse, bitten by another individual or animal that may be infected with HIV, intravenous drug user, and the like. Additional examples of individuals suspected of being exposed to HIV include, without limitation, an individual who believes that they are in need of preventive treatment. In some embodiments, an individual suspected of being infected with HIV has received an organ transplant, tissue transplant, skin graft, and the like. from another individual. The individual from which the organs, tissues, skin, etc, originated from may have not been tested for the presence and/or absence of HIV or the reliability of the test results may be in question.

p38 Inhibitor Activities and Use with HIV Infection

HIV Nef is a viral protein that interacts with host cell signal transduction proteins to provide for long-term survival of infected T cells and for the destruction of non-infected T cells by inducing apoptosis. Nef upregulates the expression of the Fas Ligand (FasL). When the infected T cells that express FasL come into contact with non-infected T cells, the FasL interacts with Fas on the non-infected cells and induces them to undergo apoptosis. The induction of apoptosis in non-infected T cells results in the specific reduction of T cells available to clear HIV infection as well as the general reduction of T cells in HIV infected patients leading to and characteristic of AIDS.

It has been discovered that p38 inhibitors can inhibit FasL expression, particularly that FasL expression believed to be due to Nef-mediated upregulation. Accordingly, p38 inhibitors can be used in methods of treating individuals who have been infected with HIV as well as treating individuals exposed to or suspected of being exposed to HIV.

It has also been discovered that p38 inhibitors inhibit HIV replication in HIV infected cells without inhibiting of T cell activation. That is, p38 inhibitors that do not inhibit T cell activation can be used to inhibit HIV replication. Accordingly, p38 inhibitors that do not inhibit T cell activation can be used in methods of treating individuals who have been infected with HIV as well as methods of treating individuals exposed to or suspected of being exposed to HIV.

In some embodiments, methods are provided for inhibiting FasL expression in cells infected with HIV. The methods comprise the step of delivering to the infected cell a p38 inhibitor in an amount sufficient to inhibit expression of FasL. In some embodiments, methods are provided for inhibiting HIV replication in cells infected with HIV. The methods comprise the step of delivering to the infected cell a p38 inhibitor in an amount sufficient to inhibit HIV replication.

The present invention additionally provides methods of screening compounds for anti-HIV activity. The methods provide testing compounds to determine their effect on FasL expression in cells that have Nef mediated upregulated of FasL expression.

The present invention additionally provides methods of screening compounds for p38 pathway inhibitory activity. The methods provide testing compounds to determine their effect on FasL expression in cells that have Nef mediated upregulated of FasL expression. Such compounds are candidate p38 pathway inhibitors which may be subsequently tested directly on p38 for p38 inhibitory activity.

JNK Inhibitor Activities and Use with HIV Infection

The present invention additionally provides methods of screening compounds for JNK pathway inhibitory activity. The methods provide testing compounds to determine their effect on FasL expression in cells that have Nef mediated upregulated of FasL expression. Such compounds are candidate JNK pathway inhibitors which may be subsequently tested directly for JNK inhibitory activity. Such compounds may be useful in treating individuals infected with or suspected of having been exposed to HIV.

p38 Inhibitors

There are many examples of p38 inhibitors in the art. U.S. Pat. Nos. 5,965,583, 6,040,320, 6,147,096, 6,214,830, 6,469,174, 6,521,655, which are each incorporated herein by reference, disclose compounds that are p38 inhibitors. U.S. Pat. Nos. 6,410,540, 6,476,031 and 6,448,257, which are each incorporated herein by reference, also disclose compounds that are p38 inhibitors. Similarly, U.S. Pat. Nos. 6,410,540, 6,479,507 and 6,509,361, which are each incorporated herein by reference, disclose compounds that are asserted to be p38 inhibitors. U.S. Published Application Nos. 20020198214 and 20020132843, which are each incorporated herein by reference, disclose compounds that are asserted to be p38 inhibitors.

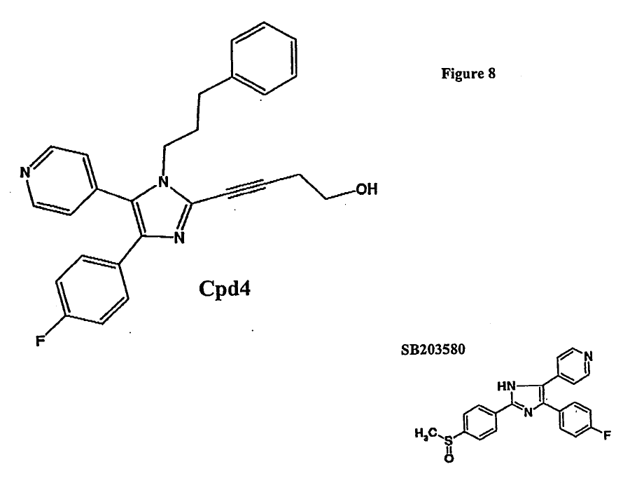

In some embodiments the p38 inhibitor is the compound disclosed in Example 4 of U.S. Pat. No. 6,521,655: 4-(4-Fluorophenyl)-2-(4-hydroxybutyn-1-yl)-1-(3-phenylpropyl)-5-(4-pyridyl)imidazole, (also named 4-[4-(4-Fluoro-phenyl)-1-(3-phenyl-propyl)-5-pyridin4-yl-1H-imidazol-2-yl]-but-3-yn-1-ol and 4-[4-(4-fluorophenyl)-1-(3-phenylpropyl)-5-(4-pyridinyl)-1H-imidazol-2-yl]-3-butyn-1-ol), which is designated Cpd. 4 in the patent and referred to herein as Cpd4. Another example of a p38 inhibitor is SB203580, which is: 4-[5-(4-Fluoro-phenyl)-2-(4-methanesulfinyl-phenyl)-3H-imidazol4-yl]-pyridine. The structures of these compounds are set forth in FIG. 8.

Treatment of EHIV Infected Individuals with p38 Inhibitors

Several embodiments of the invention include the use of p38 inhibitors to treat individuals who have been identified as having been infected with HIV.

Embodiments of the present invention are particularly useful to treat individuals who have been diagnosed as being infected with HIV. In some embodiments, methods for treating an individual who has been infected with HIV comprise the step of administering to an individual who has been identified as having been infected with HIV, an amount of a p38 inhibitor effective to inhibit expression of FasL in HIV infected T cells. The p38 inhibitor must do so by inhibiting the Nef mediated upregulation of FasL expression. By inhibiting FasL expression, the HIV infected T cells have less FasL and thereby induce apoptosis in fewer non-infected T cells. This reduction in apoptosis in non-infected T cells results in higher T cell counts including T cells involved in the recognition, attack and elimination of HIV infected cells. In some embodiments, the p38 inhibitor is effective to inhibit FasL expression levels by 50% in greater than 50% of cells in an in vitro assay at a concentration of less than 1 mM. In some embodiments, the p38 inhibitor is effective to inhibit FasL expression levels by 50% in greater than 50% of cells in an in vitro assay at a concentration of less than 0.1 mM. In some embodiments, the p38 inhibitor is effective to inhibit FasL expression levels by 50% in greater than 50% of cells in an in vitro assay at a concentration of less than 0.05 mM. In some embodiments, the p38 inhibitor is effective to inhibit FasL expression levels by 50% in greater than 50% of cells in an inz vitro assay at a concentration of less than 0.01 mM. In some embodiments, the p38 inhibitor is delivered in an amount effective to inhibit HIV replication. In some embodiments, the p38 inhibitor is effective to inhibit HIV replication by 50% as calculated in an in vitro assay at a concentration of less than 1 mM. In some embodiments, the p38 inhibitor is effective to inhibit HIV replication by 50% as calculated in an in vitro assay at a concentration of less than 0.1 mM. In some embodiments, the p38 inhibitor is effective to inhibit HIV replication by 50% as calculated in an in vitro assay at a concentration of less than 0.05 mM. In some embodiments, the p38 inhibitor is effective to inhibit HIV replication by 50% as calculated in an in vitro assay at a concentration of less than 0.01 mM. In some embodiments, the p38 inhibitor is delivered in an amount that does not inhibit T cell activation. In some embodiments, the p38 inhibitor does not inhibit T cell activation in 50% of T-cells by more than 10% as calculated in an in vitro assay at a concentration of less than 1 mM. In some embodiments, the p38 inhibitor does not inhibit T cell activation in 50% of T-cells by more than 10% as calculated in an in vitro assay at a concentration of less than 0.1 mM. In some embodiments, the p38 inhibitor does not inhibit T cell activation in 50% of T-cells by more than 10% as calculated in an in vitro assay at a concentration of less than 0.05 mM. In some embodiments, the p38 inhibitor does not inhibit T cell activation in 50% of T-cells by more than 10% as calculated in an in vitro assay at a concentration of less than 0.01 mM.

According to some embodiments the present invention provides methods for treating an individual infected with IRV comprising the step of administering an amount of a pharmaceutical composition comprising a p38 kinase inhibitor effective to inhibit expression of FasL expression. In some embodiments, the amount of a p38 kinase inhibitor administered is effective to inhibit HIV replication. In some embodiments, the amount of a p38 kinase inhibitor administered is effective to inhibit HIV replication without inhibiting T cell activation. In some embodiments, the present invention provides for methods for treating individuals who have been identified as having been infected with HIV comprising the step of administering a p38 kinase inhibitor in an amount effective to inhibit FasL expression and HIV replication. In some embodiments, the present invention provides for methods for treating individuals who have been identified as having been infected with HIV comprising the step of administering a p38 kinase inhibitor in an amount effective to inhibit FasL expression and HIV replication without inhibiting T cell activation.

The effective treatment of patients with HIV would lead to a reduction in the severity of the infection or symptoms therefrom. Stabilization or increase in T cell number is one benchmark that may be used to measure effectiveness of treatment for some patients. Another benchmark that may be used is stabilization or decrease in or elimination of viral titer. The effective treatment of previously uninfected individuals who have been or suspect that they have been exposed to HIV would be the absence of any indication of infection such as the absence of viral antigens or the absence of detectable virus.

In some embodiments, methods for treating an individual who has been infected with HIV comprise the additional step of administering to an individual who has been identified as having been infected with HIV, one or more additional therapeutics that may be used for the treatment of HIV in combination with a p38 inhibitor. Examples of “additional therapeutics” that can be used to treat HIV include, but not limited to, fusion inhibitors (i.e. enfuvirtide), nonnucleoside reverse transcriptase inhibitors (NNRTIs, i.e. delavirdine, efavirenz, nevirapine), nucleoside/nucleotide reverse transcriptase inhibitors (NRTIs, i.e. abacavir, combination of abacavir, larnivudine, and zidovudine, didanosine, lamivudine, combination of lamivudine and zidovudine, stavudine, tenofovir DF, zalcitabine, zidovudine), protease inhibitors (i.e. amprenavir, indinavir, combination of lopinavir and ritonavir, nelfinavir, ritonavir, saquinavir, invirase), and the like. Other additional therapeutics that are not described herein can also be co-administered with a p38 kinase inhibitor. The co-administration of therapeutics can be sequential in either order or simultaneous. Or any other regimen in which two or more therapeutics including a p38 inhibitor are administered in combination or conjunction.

Identification of HIV Infected Individuals

The present invention is not limited to any means for identifying the individual as infected with HIV. There are many well know methods for identifying HIV infected individuals. Once identified, p38 inhibitor is administered to the HIV infected individual in an amount effective to inhibit expression of FasL in HIV infected T cells and/or HIV replication. In some embodiments, the methods comprise the step of identifying the individual while in other, the individual may be previously diagnosed and is known to an individual who has been identified as having HIV infection.

Treatment of Individuals Exposed to HIV with p38 Inhibitors

The present invention also provides for methods to treating an individual suspected of being exposed to HIV. Many individuals who have not been diagnosed as being HIV positive are put in circumstances where it is possible that they could have possibly been exposed to HIV, but are unsure if they have been exposed to or infected with HIV. To treat an individual who is suspected of having been exposed to HIV, the individual is administered p38 inhibitors as described above in the treatment of HIV infected individuals. The treatment of individuals suspected of being exposed to HIV may include the administration of additional therapeutics as described above. The course of prophylactic treatment may be performed in conjunction with periodic monitoring for indications of HIV infection.

Pharmaceutical Compositions and Routes of Administration

The pharmaceutical composition may be formulated by one having ordinary skill in the art with compositions selected depending upon the chosen mode of administration. Suitable pharmaceutical carriers are described in the most recent edition of Remington's Pharmaceutical Sciences, A. Osol, a standard reference text in this field.

Administering the pharmaceutical composition can be effected or performed using any of the various methods known to those skilled in the art. Systemic formulations include those designed for administration by injection, e.g. subcutaneous, intravenous, intramuscular, intrathecal or intraperitoneal injection, as well as those designed for transdermal, transmucosal, oral or pulmonary administration.

For injection, the compounds of the invention may be formulated in aqueous solutions, preferably in physiologically compatible buffers such as Hanks's solution, Ringer's solution, or physiological saline buffer. The solution may contain formulatory agents such as suspending, stabilizing and/or dispersing agents. Injectables are sterile and pyrogen free. The compounds may be in powder form for constitution with a suitable vehicle, e.g., sterile pyrogen-free water, before use. For transmucosal administration, penetrants appropriate to the barrier to be permeated are used in the formulation. Such penetrants are generally known in the art.

For parenteral administration, the p38 inhibitor can be, for example, formulated as a solution, suspension, emulsion or lyophilized powder in association with a pharmaceutically acceptable parenteral vehicle. Examples of such vehicles are water, saline, Ringer's solution, dextrose solution, 5% human serum albumin, Ringer's dextrose, dextrose and sodium chloride, lactated Ringer's and fixed oils, polyethylene glycol, polyvinyl pyrrolidone, lecithin, arachis oil or sesame oil. Liposomes and nonaqueous vehicles such as fixed oils may also be used. The vehicle or lyophilized powder may contain additives that maintain isotonicity (e.g., sodium chloride, mannitol) and chemical stability (e.g., buffers and preservatives). The formulation is sterilized by commonly used techniques. Parenteral dosage forms may be prepared using water or another sterile carrier. For example, a parenteral composition suitable for administration by injection is prepared by dissolving 1.5% by weight of active ingredient in 0.9% sodium chloride solution. Alternatively, the solution can be lyophilised and then reconstituted with a suitable solvent just prior to administration.

Pharmaceutically acceptable carriers are well known to those skilled in the art and include, but are not limited to, 0.01-0.1 M and preferably 0.05 M phosphate buffer or 0.8% saline. Intravenous carriers include fluid and nutrient replenishers, electrolyte replenishers such as those based on Ringer's dextrose, and the like. Additionally, such pharmaceutically acceptable carriers can be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, ethanol, alcoholic/aqueous solutions, glycerol, emulsions or suspensions, including saline and buffered media.

The pharmaceutical compositions can be prepared using conventional pharmaceutical excipients and compounding techniques. Oral dosage forms may be elixers, syrups, tablets, pills, dragees, capsules, liquids, gels, syrups, slurries, suspensions and the like, for oral ingestion by a patient to be treated. The typical solid carrier may be an inert substance such as lactose, starch, glucose, cellulose preparations such as maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP); granulating agents; binding agents, magnesium sterate, dicalcium phosphate, mannitol and the like. A composition in the form of a capsule can be prepared using routine encapsulation procedures. For example, pellets containing the active ingredient can be prepared using standard carrier and then filled into a hard gelatin capsule; alternatively, a dispersion or suspension can be prepared using any suitable pharmaceutical carrier(s), for example, aqueous gums, celluloses, silicates or oils and the dispersion or suspension then filled into a soft gelatin capsule. Typical liquid oral excipients include ethanol, glycerol, glycerine, non-aqueous solvent, for example, polyethylene glycol, oils, or water with a suspending agent, preservative, flavoring or coloring agent and the like. All excipients may be mixed as needed with disintegrants, diluents, lubricants, and the like using conventional techniques known to those skilled in the art of preparing dosage forms. If desired, disintegrating agents may be added, such as the cross-linked polyvinylpyrrolidone, agar, or alginic acid or a salt thereof such as sodium alginate. If desired, solid dosage forms may be sugar-coated or enteric-coated using standard techniques. For oral liquid preparations such as, for example, suspensions, elixirs and solutions, suitable carriers, excipients or diluents include water, glycols, oils, alcohols, etc. Additionally, flavoring agents, preservatives, coloring agents and the like may be added.

For buccal administration, the compounds may take the form of tablets, lozenges, and the like formulated in conventional manner. The compounds may also be formulated in rectal or vaginal compositions such as suppositories or enemas. A typical suppository formulation comprises a binding and/or lubricating agent such as polymeric glycols, glycerides, gelatins or cocoa butter or other low melting vegetable or synthetic waxes or fats. For administration by inhalation, the compounds for use according to the present invention are conveniently delivered in the form of an aerosol spray from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

The formulations may also be a depot preparation which can be administered by, implantation (for example subcutaneously or intramuscularly) or by intramuscular injection. In such embodiments, the compounds may be formulated with suitable polymeric or hydrophobic materials (for example as an emulsion in an acceptable oil) or ion exchange resins, or as sparingly soluble derivatives, for example, as a sparingly soluble salt.

Alternatively, other pharmaceutical delivery systems may be employed. Liposomes and emulsions are well known examples of delivery vehicles that may be used. Certain organic solvents such as dimethylsulfoxide also may be employed, although usually at the cost of greater toxicity. Additionally, the compounds may be delivered using a sustained-release system, such as semipermeable matrices of solid polymers containing the therapeutic agent Various of sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

The compounds used in the invention may also be formulated for parenteral administration by bolus injection or continuous infusion and may be presented in unit dose form, for instance as ampoules, vials, small volume infusions or pre-filled syringes, or in multi-dose containers with an added preservative.

Preservatives and other additives can also be present, such as, for example, antimicrobials, antioxidants, chelating agents, inert gases and the like. All carriers can be mixed as needed with disintegrants, diluents, granulating agents, lubricants, binders and the like using conventional techniques known in the art.

Dosages and Treatment Regimens

According to the present invention, methods of treating an individuals who has been identified as having been infected with HIV are performed by delivering to such individuals an amount of a p38 inhibitor sufficient to inhibit of FasL expression in cells infected with HIV. By doing so, the infected cells will induce apoptosis in fewer uninfected cells that they come into contact with and thereby the number of T cells will increase or be reduced at a slower rate. Thus, patient survival may be extended and/or quality of life improved as compared to treatment that does not include p38 inhibitor administration in doses that inhibit of FasL expression. The present invention provides for methods of inhibiting Nef mediated upregulation of FasL expression in HIV infected cells comprising the step of delivering p38 inhibitor to such cells in an amount effective to inhibit Nef mediated upregulation of FasL expression.

HIV Replcation

The pharmaceutical compositions described above may be administered by any means that enables the active agent to reach the agents site of action in the body of the individual. The dosage administered varies depending upon factors such as: pharmacodynamic characteristics; its mode and route of administration; age, health, and weight of the recipient, nature and extent of symptoms; kind of concurrent treatment; and frequency of treatment.

The amount of compound administered will be dependent on the subject being treated, on the subject's weight, the severity of the affliction, the manner of administration and the judgment of the prescribing physician. In some embodiments, the dosage range would be from about 1 to 3000 mg, in particular about 10 to 1000 mg or about 25 to 500 mg, of active ingredient, in some embodiments 1 to 4 times per day, for an average (70 kg) human. Generally, activity of individual compounds used in the invention will vary.

Dosage amount and interval may be adjusted individually to provide plasma levels of the compounds which are sufficient to maintain therapeutic effect. Usually, a dosage of the active ingredient can be about 1 microgram to 100 milligrams per kilogram of body weight. In some embodiments a dosage is 0.05 mg to about 200 mg per kilogram of body weight. In another embodiment, the effective dose is a dose sufficient to deliver from about 0.5 mg to about 50 mg. Ordinarily 0.01 to 50 milligrams, and in some embodiments 0.1 to 20 milligrams per kilogram per day given in divided doses 1 to 6 times a day or in sustained release form is effective to obtain desired results. In some embodiments, patient dosages for administration by injection range from about 0.1 to 5 mg/kg/day, preferably from about 0.5 to 1 mg/kg/day. Therapeutically effective serum levels may be achieved by administering multiple doses each day. Treatment for extended periods of time will be recognized to be necessary for effective treatment.

In some embodiments, the route may be by oral administration or by intravenous infusion. Oral doses generally range from about 0.05 to 100 mg/kg, daily. Some cqmpounds used in the invention may be orally dosed in the range of about 0.05 to about 50 mgkg daily, while others may be dosed at 0.05 to about 20 mg/kg daily. Infusion doses can range from about 1.0 to 1.0.times.10⁴ microgram/kg/min of inhibitor, admixed with a pharmaceutical carrier over a period ranging from several minutes to several days.

Drug Discovery Methods

The present invention additionally provides methods of screening compounds for anti-HIV activity. The methods provide testing compounds to determine their effect on FasL expression in cells that have Nef mediated upregulated of FasL expression. FasL expression can be used as a marker for identifying compounds. According to some embodiments, the methods for identifying such compounds comprise performing a test assay that comprises the steps of contacting a cell that expresses Nef in sufficient quantities to upregulate expression of FasL with a test compound and measuring the level of FasL expression. The level of FasL expression in the test assay is compared to the level of FasL expression that occurs in the absence of the test compound. In some embodiments the method further comprises a negative control assay that comprises the steps of contacting a cell that expresses Nef in sufficient quantities to upregulate expression of FasL with a sample free of any material that modulates FasL expression and measuring the level of FasL expression. The negative assay data may be used a reference point in comparison with the test assay data. In some embodiments the method further comprises a positive control assay that comprises the steps of contacting a cell that expresses Nef in sufficient quantities to upregulate expression of FasL with a compound that is known to inhibit Nef mediated FasL expression and measuring the level of FasL expression. The positive assay data may be used a reference point in comparison with the test assay data. The methods can be performed using cells infected with HIV or cells engineered to express Nef. Cells useful in such assays undergo Nef mediation upregulation of FasL expression. Assays that can be used for the methods to measure FasL expression are well known to those of ordinary skill in the art and require only routine experimentation. Examples of assays that are well known to those of ordinary skill in the art include ELISA, Sandwich Assays, flow cytometry, immunoprecipitation, PCR and the like.

In some embodiments, kits may be provided for performing such assays. Kits comprise a) either: 1) a container comprising an expression vector that encodes Nef for transfection into suitable cells and optionally cells which can be used or 2) transformed cells that express Nef or 3) both 1) and 2); and b) instructions for performing the assay. The kit may further comprise reagents useful in the detection of FasL expression. The kit may further comprise photographs, examples and/or depictions of positive and negative data.

According to certain aspects of the invention, methods of identifyig compounds that inhibit Nef-mediated activation of the p38 kinase and/or JNK signaling pathways are provided. Methods to measure the level of activation of the p38 pathway include, but are not limited to, determining the level of phosphorylation of p38 or JNK, of molecules downstream of p38 or JNK, including but not limited to Mitogen-activated protein kinase-activated protein kinase-2 (MAPKAPK-2, MK2), heat shock protein-70 (HSP-70), ATF-2, or c-jun or of molecules upstream of p38 or JNK, such as MKK3, MKK4, MKK6 or others.”

The present invention additionally provides methods of screening compounds for inhibition of p38 and the p38 pathway. The methods provide testing compounds to determine their effect on FasL expression in cells that have Nef mediated upregulated of FasL expression. FasL expression can be used as a marker for identifyig compounds. According to some embodiments, the methods for identifying such compounds comprise performing a test assay that comprises the steps of contacting a cell that expresses Nef in sufficient quantities to upregulate expression of FasL with a test compound and measuring the level of FasL expression. The level of FasL expression in the test assay is compared to the level of FasL expression that occurs in the absence of the test compound. In some embodiments the method further comprises a negative control assay that comprises the steps of contacting a cell that expresses Nef in sufficient quantities to upregulate expression of FasL with a sample free of any material that modulates FasL expression and measuring the level of FasL expression. The negative assay data may be used a reference point in comparison with the test assay data. In some embodiments the method further comprises a positive control assay that comprises the steps of contacting a cell that expresses Nef in sufficient quantities to upregulate expression of FasL with a p38 inhibitor and measuring the level of FasL expression. The positive assay data may be used a reference point in comparison with ihe test assay data. The methods can be performed using cells infected with HIV or cells engineered to express Nef. Cells useful in such assays undergo Nef mediation upregulation of FasL expression. Assays that can.be used for the methods to measure FasL expression are well known to those of ordinary skill in the art and require only routine experimentation. Examples of assays that are well known to those of ordinary skill in the art include ELISA, Sandwich Assays, flow cytometry, immunoprecipitation, PCR and the like. Methods may further comprise steps to further test the activity of the compounds on p38.

In some embodiments, kits may be provided for performing such assays. Kits comprise a) either: 1) a container comprising an expression vector that encodes Nef for transfection into suitable cells and optionally cells which can be used or 2) transformed cells that express Nef or 3) both 1) and 2); and b) instructions for performing the assay. The kit may further comprise reagents useful in the detection of FasL expression. Kits may optionally include a container comprising a p38 inhibitor The kit may further comprise photographs, examples and/or depictions of positive and negative data. Additionally, kits may comprise components to further test compounds for their effect on p38 activity.

The present invention additionally provides methods of screening compounds for inhibition of JNK and the JNK pathway. The methods provide testing compounds to determine their effect on FasL expression in cells that have Nef mediated upregulated of FasL expression. FasL expression can be used as a marker for identifyng compounds. According to some embodiments, the methods for identifying such compounds comprise performing a test assay that comprises the steps of contacting a cell that expresses Nef in sufficient quantities to upregulate expression of FasL with a test compound and measuring the level of FasL expression. The level of FasL expression in the test assay is compared to the level of FasL expression that occurs in the absence of the test compound. In some embodiments the method further comprises a negative control assay that comprises the steps of contacting a cell that expresses Nef in sufficient quantities to upregulate expression of FasL with a sample free of any material that modulates FasL expression and measuring the level of FasL expression. The negative assay data may be used a reference point in comparison with the test assay data. In some embodiments the method further comprises a positive control assay that comprises the steps of contacting a cell that expresses Nef in sufficient quantities to upregulate expression of FasL with a JNK inhibitor and measuring the level of FasL expression. The positive assay data may be used a referenceipoint in comparison with the test assay data. The methods can be performed using cells infected with HIV or cells engineered to express Nef. Cells useful in such assays undergo Nef mediation upregulation of FasL expression. Assays that can be used for the methods to measure FasL expression are well known to those of ordinary skill in the art and require only routine experimentation. Examples of assays that are well known to those of ordinary skill in the art include ELISA, Sandwich Assays, flow cytometry, immunoprecipitation, PCR and the like. Methods may further comprise steps to further test the activity of the compounds on JNK.

In some embodiments, kits may be provided for performing such assays. Kits comprise a) either: 1) a container comprising an expression vector that encodes Nef for transfection into suitable cells and optionally cells which can be used or 2) transformed cells that express Nef or 3) both 1) and 2); and b) instructions for performing the assay. The kit may further comprise reagents useful in the detection of FasL expression. Kits may optionally include a container comprising a JNK inhibitor The kit may further comprise photographs, examples and/or depictions of positive and negative data. Additionally, kits may comprise components to further test compounds for their effect on JNK activity.

### EXAMPLES

### Example 1

A novel p38 inhibitor has been observed to undermine HIV-1 replication iin vitro. Divergent virus isolates on multiple cell phenotypes were all susceptible to inhibition by p38 blockade. The invention relates to the protective effects of p38 blockade on HIV pathogenesis. The p38 blockade can to a great extent prevent HIV mediated apoptosis of target cells. This apoptotic event was coincident with the upregulation of FasL by HIV infection. Analysis demonstrated that p38 blockade could prevent FasL upregulation. Analysis of the effects of individual gene products of HIV clearly indicated that Nef was uniquely responsible for this up regulation, and that Cpd4 could prevent Nef driven FasL activation which was observed to be-dependent on p38 linked transcription factor activation. Furthermore, Cpd4 could prevent Nef driven apoptosis of bystander cells. There is a link between HIV-1 nef driven pathogenesis, activation of the p38 MAPK pathway and host cell apoptosis. Preventing exploitation of this pathway by HIV-1 represents a likely important and readily accessible new area for HIV drug development.

### Materials and Methods

MAPK Inhibitor (p38 Inhibitors)

The p38 inhibitors Cpd4 or SB203580 have been previously described. Both compounds inhibit p38 MAP Kinase, at 1 μM concentrations. The compounds were dissolved in dimethylsulfoxide (DMSO) and diluted to give a final concentration of 100 μM or 1 μM as indicated. As a control an equal concentration of DMSO was added to the experimental control cells.

Cells

The human CD4⁺ T cell line Jurkat, or the monocyte line U937 were obtained from the American Type Culture Collection (Rockville, Md.). Cells were passaged in RPMI1640 (Gibco-BRL, MD) supplemented with 10% FBS, 2 mM L-glutamine, 100 U/ml penicillin G, and 100 μg/ml streptomycin maintained at 37° C. and 5% CO₂ and verified routinely to certify that they were Mycoplasma negative. Human PBMCs were isolated from healthy HIV-seronegative donors by Ficoll-Hypaque separation (Pharmacia Biotech AB, Sweden). Peripheral blood mononuclear cells (PBMCs) from healthy adults were incubated for 2 days with 5 μg/ml phytohemagglutinin (PHA, Sigma) before the addition of 5 U/ml of human recombinant interleukin-2 (hrIL-2; R&D system, MN).

Monocyte-derived macrophages (MDM) were prepared from PBMCs obtained from healthy donors and incubated at 37° C. in polystyrene T-75 flasks for 4-10 hrs at 37° C. After the incubation the cells were washed with RPMI 1640 for 3 times to remove non-adherent cells. The adherent monocytes were detached with ethylene diamine tetra-acetic acid (EDTA) and the purity of the monocyte cell populations thus isolated was >98% as determined by FACS staining for CD14⁺. The CD14⁺ positive cells were incubated in 6-well plates at a density of 1×10⁶ cells/ml in RPMI medium supplemented with 10% human serum.

Construction and Generation of HIV-1 Virions Packaged with Nef

Constructs containing Nef were generated using overlap extension PCR at the indicated codons and were cloned into the pCDNA3.1 vector (InVitrogen, CA). The HIV-1 proviral infectious vector pNL4-3 was obtained through the NIH AIDS research and Reference Reagent Program. This vector has been rendered Nef deficient by 5′ frame shift mutation in the Nef region (He, J., Choe, et al, J. Virol. 69, 6705-6711 (1995) which is incorporated herein by reference).

To prepare infectious viral stocks, HIV-1 proviral DNA (pNL4-3 Wt and pNL4-3 and Nef delta) were generated as described Muthumani, K., et al, Journal of Biol. Chem. 277: 37820-37831 (2002). The viral titers were determined by infection of the human T cell line Jurkat using serially diluted virus supernatant. Typically, viral titers had a range of 5-10×10⁶ infectious units (ifu)/ml. p24^{gag} antigen was measured by capture ELISA (Coulter, Fla.). Viral stocks were normalized for virus content by infection and titration and stocks were stored in the presence of 10% FBS in aliquots at −80° C. until their use.

HIV-1 Viruses and Infection

Viruses that were assayed in these studies included the pNL 4-3 (dual tropic) virus, which uses both CCR5 & CxCR4 receptors, and 89.6 (dual tropic), which uses CCR5 & CxCR4 receptors. In addition clade specific viruses were studied and these were obtained through the NIH AIDS research and Reference Reagent Program. For infection studies human PBMCs were isolated from normal, sero-negative donors, and infection was accomplished by incubating target cells with HIV-1 virus at a concentration of 100 TCID₅₀/10⁶ cells/ml. Culture supernatant was collected at 6, 12 and 24 hr intervals and assayed for virus production by measuring the p24 antigen released into the medium by ELISA (Coulter, FL) according to the manufacturer's instructions. Some data are presented as mean±SEM. For data presented as percent change, the base line (medium alone) value was subtracted from the value of each experimental condition as described in the legend.

Apoptosis and Caspase Studies

FACS analysis was performed to identify cells undergoing apoptosis. Equal numbers of cells from each test group were collected for analysis. Apoptosis in experimental cells was analyzed by using an Annexin-V assay kit from PharMingen (CA). Data was analyzed by the CELL Quest program (Beckton-Dickinson, CA). Caspase 3 activity was determined using Caspase-3/CPP32 colorimetric protease assay kit according to the manufacturer's instructions (MBL, Nagoya, Japan).

CD95L Analysis by Flow Cytometry

Single cell suspensions were washed in PBS (pH 7.2) containing 0.2% bovine serum albumin and 0.1% NaN₃. Cells were incubated with PE labeled □-CD95L antibody (NOK-1, 0.05 μg/ml, BD Pharmingen, CA, USA) antibody for 60 minutes at 4° C. Cells were washed with PBS and fixed with 2% paraformaldehyde and resuspended in 200 μl PBS and subjected to FACS (FL2 histogram) analysis (Beckton Dickinson, CA). Data were analyzed using the CELL Quest program (Beckton-Dickinson, CA).

Immunoblot Analysis

Experimental cells were washed with ice-cold PBS, and the cells were lysed in protein lysis buffer (20 mM Tris (pH7.4), 150 mM NaCl, 1 mM EDTA, 1 mMEGTA, 1% triton, 2.5 mM sodium pyrophosphate, 1 mM □-glycerolphasphate, 1 mM Na₃Vp4, 1 μM/ml leupeptin and 1 mM phenyl methyl-sulfonyl fluoride). After a brief sonication, the lysates were clarified by centrifugation at 10,000 rpm and protein content was measured by the Bradford method (Bio-Rad, CA). Nuclear extracts were prepared using NE-PER nuclear and cytoplasmic extraction reagents (Pierce, USA) and the protein concentration of the nuclear extract was determined by BCA-200 protein assay kit (Pierce, USA) assayed following the manufacturer's instructions.

For Western blot analysis 50 μg of protein per lane was loaded onto acrylamide gels and separated by 12% SDS-PAGE and blotted to PVDF tmnsfer membrane. Immunoblot was carried out using the primary antibody (c-Jun, Phospho-p38 MAP Kinase (Thr180/Tyr182); Cell Signaling, MA, USA) and detected with secondary HRP-conjugated anti-rabbit IgG using Amersham ECL system. Further, the membrane was washed thoroughly using lxWestern re-probe buffer (Geno Tech, MO) and re immunoblotted with anti-actin (Calbiochem, CA) antibody, which recognizes the actin expression in cultured and serves cells as a positive control for gene expression and as an internal standard.

HIV-1 Specffic CD8⁺ T Cell Apoptosis

Human PBMC's were isolated from healthy HIV-seronegative donors as described above. Purified CD8⁺ T cells were isolated from PBMC by negative immunoselection using magnetic beads (Dynal, CA), for depletion of CD4⁺ T cells. The puity of the isolated CD8+ T cells was determined to be by flow >99% pure. MDM were purified from freshly isolated PBMC as described above. MDM cells were cultured in 6 well plates at 0.5×10⁶ cells/ml/well with 10 ng/ml M-CSF (R&D systems, MN) in RPMI 1640/10% Human serum. On day 5 MDM were infected with MRV-1 viruses 89.6 at a concentration of 100 TCID₅₀/0.5×10⁶ cells/ml/well. Different experimental groups were as follows: Group I treated as Mock (untreated); Group II treated with latex beads only, Group III treated with 20 μg/ml of neutralizing anti-FasL monoclonal antibody (ZB4, MBL, Japan) was added, Group IV was treated with latex beads and 1 μM of p38 inhibitors (Cpd4). On day 5, 0.5×10⁶ polystyrene latex beads (IDC Spheres, OR) were added to groups II, III and IV for 2 hr at 37° and incubated in a CO₂ incubator. Anti-FasL monoclonal antibody was added for 90 min before 0.5×10⁶ autologous purified CD8⁺ T cells were added. After 14 hrs of incubation, cells were harvested and stained with Mab's including anti CD8 (clone RPA-T8; BD Pharmingen, CA) and/or CD14 (Clone M5E2; BD Pharminged, CA) and further counter stained with Annexin V-FITC (BD Pharmingen, CA). The analysis was performed on a gated CD8⁺ T-cells (R1) and further CD8⁺ positive cells were analyzed for the CD8⁺/Annexin V positive cells (R2 population) in all experimental samples to further analysis the Apoptosis.

NF-κB and AP-1 Reporter Assay

Jurkat cells (1×10⁶) were seeded onto a 60 mm culture dish (Falcon) and elctrotransfected the cells next day with NF-κB or AP-1 dependent reporter plasmid (10 μg) with pNef (10 μg), and/with or without Cpd4 (1 μM). After 24 hours, transfected cells were lysed with Reporter lysis Buffer (RLB) according to the manufacturer's instructions (Roche, USA). Luciferase activity was measured via LUMAT LB9501 (Berthold, USA). β-Gal levels were used to normalize for transfection efficiency with the chemiluminescent β-Gal reporter gene Assay kit (Roche, USA).

Statistical Analysis

All of the experiments were-performed at least three times. Results are expressed as mean±SE. Statistical comparisons were made by ANOVA followed by an unpaired two-tailed Student's t test. Ps<0.05 were considered significant.

### Results

HIV-1 Induced Apoptosis is Blocked by p38 MAPK/JNK Inhibitor

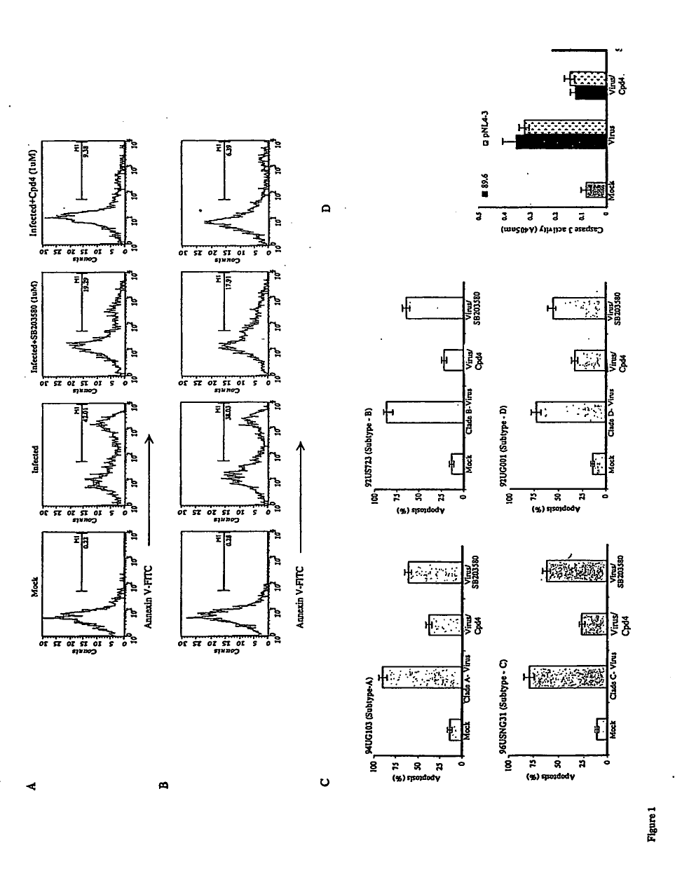

To determine the role of p38 MAPK/JNK inhibitor in HIV-1 induced apoptosis, equal numbers of human PBMCs were infected with mock (control), or 89.6 or pNL4-3 virus. In addition the infected cells were incubated 1 μM SB203580 or Cpd4 or control. Cells were collected 4 days post infection from each group and analyzed by staining with Annexin V-FITC. As shown in FIG. 1A and FIG. 1B, HIV-1 infection promoted apoptosis of target cells with either viral isolate. SB203580 inhibited apoptosis driven by virus more than 50% at this concentration. Cpd4 was even more potent at inhibiting apoptosis driven by either viral isolate reaching levels of inhibition over 80% (panel-iii & panel-iv). These results suggest that HIV-1 induced apoptosis may mediated in part through p38 MAPK activation and phosphorylation which can be ilnhibited by blocking the p38 cascade. As this activity is mediated by the virus targeting a host signaling pathway it is likely a conserved viral function. Accordingly we analyzed induction of apoptosis by 4 subtype divergent HIV viral isolates. All four primary subtype divergent viruses induced strong, clearly detectable levels of target cell apoptosis in this infection system. To confirm that the observed apoptosis was at least in part mediated through the p38 pathway we tested the ability of both inhibitors to block this viral induced event. The results were similar to those observed in FIG. 1A and FIG. 1B in that both compounds SB203580 and Cpd4 could inhibit apoptosis. Again Cpd4, which has higher anti-p38 activity, again was most potent in this inhibition again supporting a direct role for the p38 MAPK pathway in apoptosis induction (FIG. 1C).

To further confirm these observations, the late down stream activation of apoptosis induction, Caspase 3 activity, was examined. Caspase 3 is also referred to as the executioner caspase as it is downstream of both the TNF receptor driven Caspase 8 apoptotic pathway as well as the mitochondrial driven Caspase 9 mediated pathway. Caspase 3 is responsible for initiating all the end stage effects of apoptosis. Caspase 3 was activated by infection with either 89.6 or pNL4-3 virus (FIG. 1D). This activity was inhibited by alnost 50% by SB203580 and close to 80% by Cpd4. These data closely agree with the data obtained using the Annexin V stain system.

FasL Expression is Increased by HIV-1 Infection

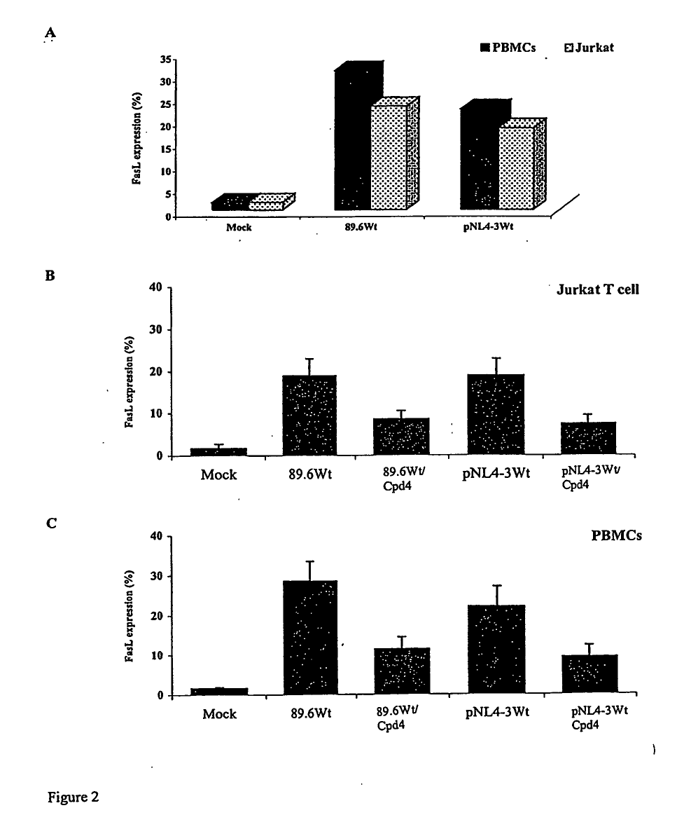

Fas (CD95) is a cell receptor that has been shown to trigger apoptosis following it's cross-linking by certain antibodies or by interaction with its ligand which can be expressed on effector T cells and NK and Dendritic cells. Previous studies indicate that HIV infected cells upregulate FasL expression and selectively induce the apoptosis of FasL susceptible T cells from HIV positive individuals. The upregulation of FasL on HIV infected cells has been reported to occur through multiple mechanisms however recently this activation has been linked to the Nef antigen. However, the pathway involved in this regulation is unknown. Based on the induction of apoptosis observed (FIG. 1C) the relevance of the p38 pathway to FasL driven apoptosis was examined. Confirmation of the induction of FasL by HIV infection was first sought. Strong induction of FasL was observed by 48 hours post infection in both T cell lines as well as PBMC's (FIG. 2A). Data indicated that the p38 MAPK and SAP/JNK cascades might be involved in HIV-1 induced apoptosis. Therefore it was reasoned that p38 inhibitors may function to in part block HIV driven upregulation of FasL. Mock or 89.6 or pNL4-3−1 infected PBMC's or Jurkat cells were treated with 1 μm of Cpd4 and FasL expression was determined by Flow cytometric analysis. Strong inhibition of FasL induction by p38 blockade was observed (FIG. 2B and FIG. 2C). This inhibition occurred in either cell phenotype and with either virus studied. These data support that the apoptosis observed is driven in part by viral activation of the p38 pathway. This activation is most likely important for driving expression of the FasL on HIV infected cells.

HIV-1 Nef and CD95L Apoptosis

HIV-1 encodes seven regulatory genes. Among these nef gene product is highly conserved. Nef appears to be a substantial viral virulence factor as has been shown to be critical for the development of AIDS in animal model systems. Furthermore, Nef appears important in human infection and pathogenesis as well. Nef has been identified to have two direct and one indirect target within the host cell. Specifically, Nef association at the plasma membrane with CD4⁺ is responsible in part for the surface modulation of this receptor on CD4⁺ T cells. In addition, and possibly by the same mechanism, Nef down modulates the expression of MHC class I antigen as well. The effect of this down modulation is thought to help the virus escape immune surveillance. Of importance to this current study is a recent report that Nef is also involved in the upregulation of FasL on infected cells. This upregulation could trigger effector T cells, which come in contact with a FasL expression cell to be eliminated thus further facilitating virus immune escape. However, the pathway used by Nef to influence FasL expression is unknown. As the p38 pathway is important for activation of several immune relevant genes, the possibility that Nef may directly activate the p38 pathway to drive FasL expression was explored. Accordingly four HIV gene products, which have all been implicated in the phenomena of host cell apoptosis, tat, vpr, env and nef, were studied to determine which if any of these individual genes could drive FasL expression. The four constructs were transfected individually into Jurkat cells and the levels of FasL expression was compared along with control plasmids.

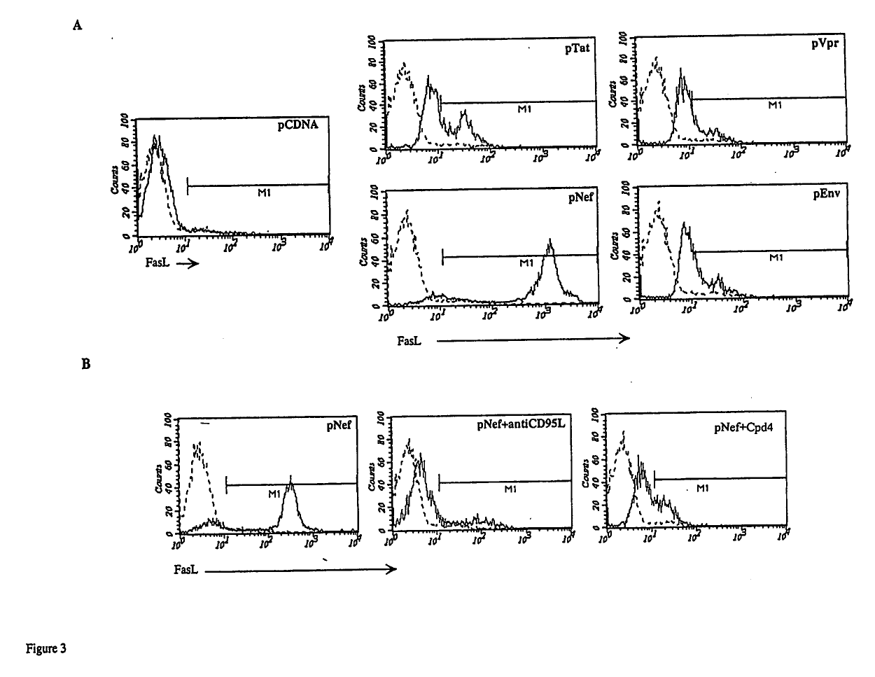

As shown in FIG. 3A Nef was the most potent inducer of FasL expression of the genes studied. While Tat, Vpr and Env all induced low level expression of FasL, transfection with Nef specifically induced high levels of induction of FasL expression. These results support a unique role for Nef in driving FasL expression during HIV infection.

Whether Nef was driving FasL expression through the p38 pathway was next tested directly. Jurkat cells were transfected with the pNef construct and treated or not with Cpd4 (FIG. 3B) and FasL expression was determined. In the absence of compound high levels of FasL expression was induced by Nef transfection. This result confirms the recent study showing a relationship between FasL induction and Nef expression. However, in the presence of Nef and Cpd4 potent inhibition of Nef driven induction of FasL was observed. This data strongly suggests that Nef uses the p38 pathway to activate FasL expression.

HIV-1 Nef Protein Sensitizes Aapoptosis Via Functional Upregulation of the CD95/CD95L Pathway

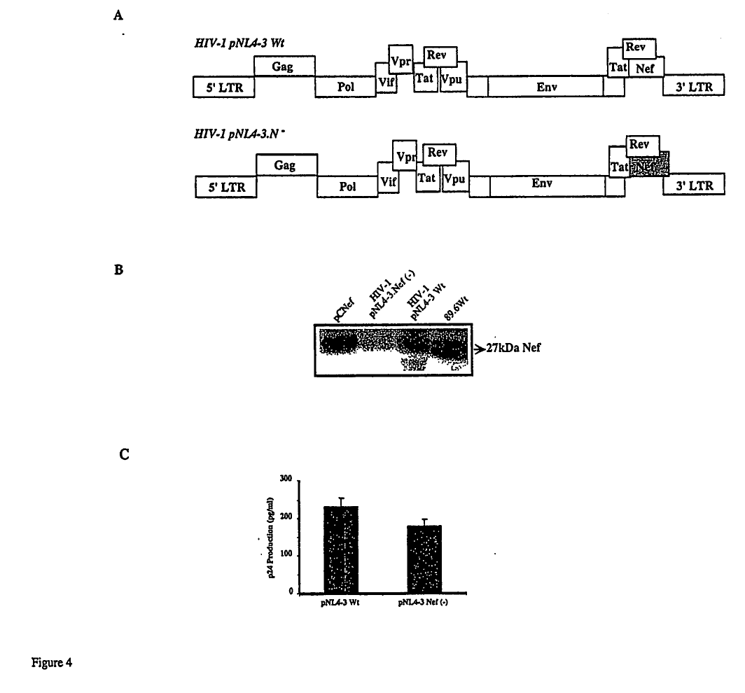

The above studies identified a function of Nef and its interaction with the p38 pathway as an isolated gene product. Confirmation of this activity in the context of viral infection was next sought. Accordingly, a set of viruses which were defective in Nef expression were constructed. Such vectors allow for testing of the Nef effect during infection. Viruses were constructed as descnbed in the materials section by introducing frame shift mutations in the Nef ORF (FIG. 4A). To confirm the Nef deletion the proviral constructs were transiently transfected into 293T cells, a human kidney-derived cell line and expression/deletion of Nef protein from transfected cells were analyzed by immunoblotting with human anti-HIV-1 Nef antibody (FIG. 4B). Only the specific deletion containing viruses lost expression of Nef as expected. Next virus particles were produced by transfection of constructs into 293T cells and p24 production was determined by ELISA. Following concentration and standardization supernatants were used to infect primary target cells and viral p24^{gag} expression was detected by FACS analysis by staining cells with anti-p24^{gag} antibody (data not shown) and also production of p24 by pNL4-3 Wt and pNL4-3 delta Nef virions was quantified by ELISA (FIG. 4C). In all cases similar levels of virus infection was observed.

FasL Expression is Increased in Infected Cells by HIV-1 Nef

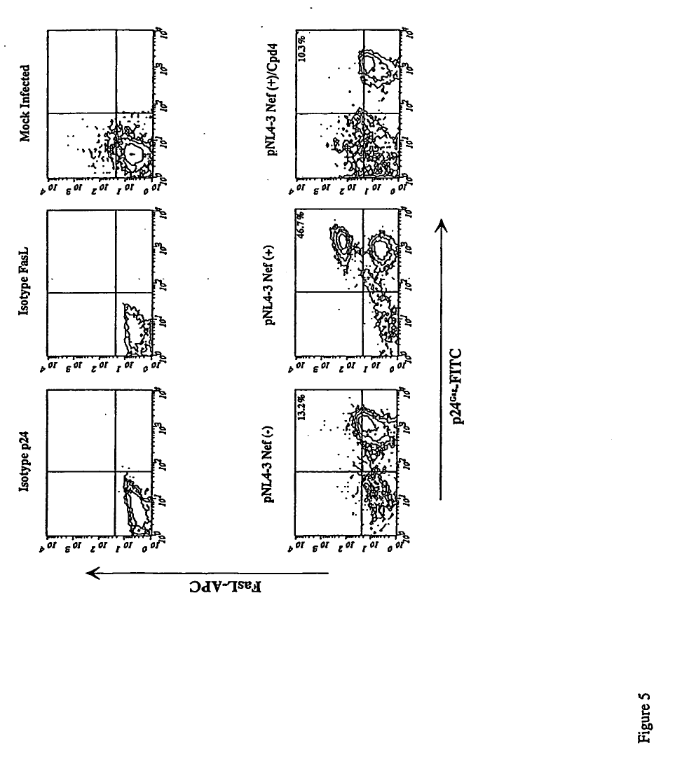

The observed upregulation of the FasL in HIV-1 infected PBMC's cultures could be the direct result of HIV infection of individual cells, or alternatively it could be the indirect result of cytokines and /or soluble viral proteins produced by HIV-infected cells. A recent study by Zauli et al. (Blood 93(3):1000-10 (1999), which is incorporated herein by reference) reported that HIV-1 Nef protein alters T-cell development and signaling and is required for optimal viral replication. Nefpositive or negative pseudoviral infection assay using the viruses constructed above was used to analyze the effect of viral borne Nef on FasL expression. Following viral infection of PBMC's, flow cytometry of intracytoplasmic p24^{gag} and FasL was determined 4 days after infection. As shown in FIG. 5, a direct correlation was observed in HIV-1 Nef positive viral infected cells which upregulated FasL expression. The percentage of dual positive cells is depicted in the upright quadrant each group. Approximately ½ of the infected cells that exhibited p24 were FasL positive on day 4. The FasL positive cells constituted a unique separate population of cells that was only present in the Nef-containing virus. The direct relationship between p24 and FasL expression was highly statistically significant (p<0.01). Interestingly, Nef mediated FasL expression was aborted completely by treatment of the culture with Cpd4 at 1 μM concentration. These results further demonstrate the link between Nef, FasL induction and the p38 pathway.

HIV Specific CD8⁺ T Cells Induced Apoptosis Blocked by p38 Inhibitor

Apoptosis induced by the Fas/FasL interaction plays a key role in the immune system through its participation in peripheral T-lymphocyte homeostasis and in lymphocyte-mediated cytotoxicity. Dysregulation of physiologic Fas/FasL interactions can result in immune disease states characterized by enhanced levels of Fas-mediated apoptosis. FasL is up regulated on HIV infected macrophages and perhaps T cells, which have been hypothesized, to be able to induce Fas mediated apoptosis of CD8⁺ effector T cells. Encounter of the susceptible effector T lymphocyte with a FasL expressing immune cell could trigger its death by apoptosis. Accumulating evidence indicates that antigen-presenting cells such as macrophages may play a key role in the elimination of activated effector T cells possibly by this mechanism. Accordingly, the possibility of macrophage mediated HIV-specific CD8⁺ T cell apoptosis induced by HIV infection and its link to the p38 pathway was investigated. Since FasL is stored intracellularly by macrophages, and is expressed on the surface or released as soluble FasL when infected macrophages are stimulated by phagocytosis or artificially by their activation, primary macrophages were stimulated with latex beads to induce their activation after infection with HIV virus.

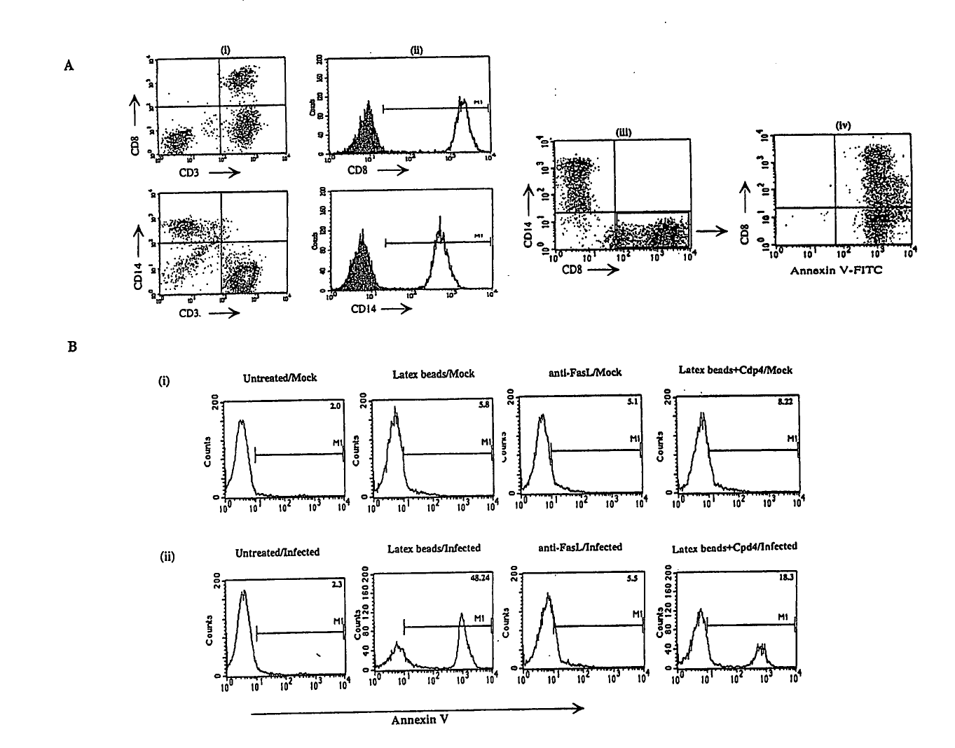

As shown in FIG. 6A, four days following infection of the culture with 89.6 at 100TCID₅₀ the CD14 population can be easily discriminated from the CD8⁺ Lymphocyte population. Furthermore, further gating on the CD8⁺ T cell population using Annexin V as a marker of apoptosis clearly shows high levels of apoptosis is occurring in the bystander CD8+ T cell population at this time. The link between the observed apoptosis to the p38 pathway was investigated. Again macrophages were infected and again gated on the CD8⁺ T cell population. As shown in FIG. 6B, uninfected macrophages, even when activated, induce little apoptosis of the CD8⁺ T cell population under any of the experimental conditions tested. In contrast, latex beads stimulated HIV infected macrophages potently drive apoptosis killing almost 50% of the CD8⁺ T cells in this culture. To determine if this apoptosis was mediated by the Fas/FasL pathway, we attempted to block the apoptosis by adding a neutraliig anti-FasL monoclonal antibody to the cell culture. FasL addition could block much of the apoptosis observed. The ability of the p38 inhibitor to impact on the observed bystander CD8⁺ T cell apoptosis was examined. The p38 blockade was almost as effective as FasL antibody at preventing bystander apoptosis but there was some difference observed. This difference is in agreement with prior reports suggesting that other accessory genes, likely through non p38 mediated mechanisms, can play a role in the bystander apoptosis induced by FasL expression. Taken together, these findings clearly show that HIV-infected macrophages can induce CD95/Fas mnediated apoptosis of CD8⁺ T cells during HIV infection. The killing of these bystander CD8⁺ T cells can almost completely be prevented by p38 blockade, illustrating the dominance of Nef in the bystander killing process.

Cpd4 Represses HIV-1 Nef Mediated Transcription of AP-1

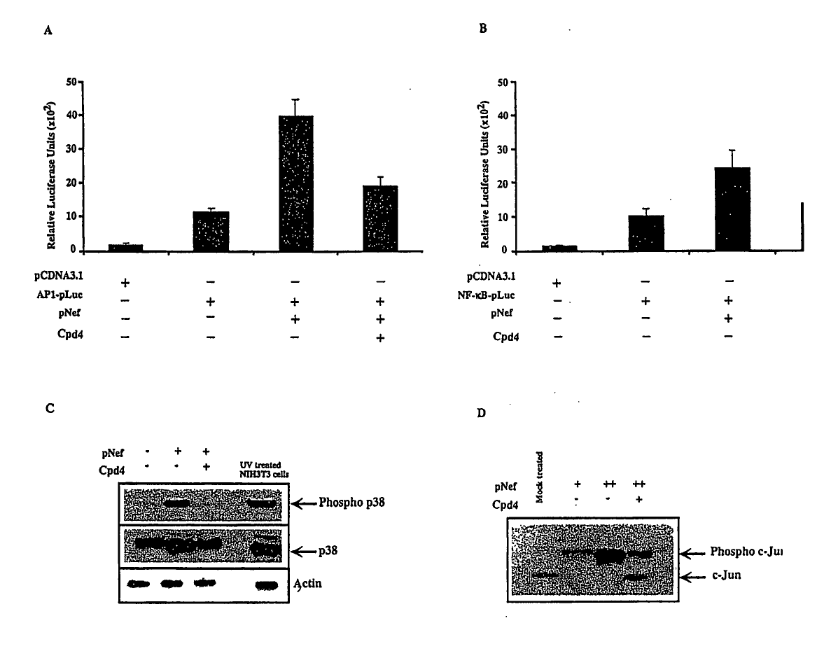

AP-1 is an important transcription factor in inmmune activation. It plays a central role in immune expansion by playing a role in cytokine production in antigen presenting cells as well as T cells. Following its discovery, AP-1 activity was found to be induced by many stimuli, including growth factors, cytokines, T cell activators, neurotransmitters, and UV irradiation. Several mechanisms are involved in induction of AP-1 activity and may be classified as those that increase the abundance of AP-1 components and those that stimulate their activity. Eukaryotic cells respond to external stresses and inflanmmatory factors through the activation of mitogen activated protein kinases (MAPK) eventiually leading to transcriptional alteration. Specifically, c-Jun N-terminal kinases (JNK) and p38 MAPK have been implicated in these responses. In most cases, p38 is activated by MAP kinase kinases (MKK) 3 and 6 through their phosphorylation. MAPK activated trnnscription is essential in driving the transcriptional activation of FasL via NF-κB and AP-1. Therefore, an investigation of the role of Nef for inducing FasL transcription through these pathways was done. First, the pNef was electrophorated into Jurkat cells with reporter vectors specific for NF-κB and AP-1 activation. As shown in FIGS. 7A and 7B, Nef effectively activated both of these transcription factors. Treatment of both groups with the p38 inhibitor Cpd4 effectively blocked this effect. However, it is interesting to note that a precursor kinase MEKK1 has been shown to effectively induce both p38/JNK and IKK activation.

HIV-1 Nef Induced Phosphorylation of p38 and JNK

The role of Nef in inducing MEKK1 remained to be determined. Next, Nef was delivered into Jurkat cells and treated them with or without the p38 MAPK inhibitor Cpd4. To investigate the HIV-1 Nef induced phosphorylation state of p38MAPK and JNK phospho-specific antibodies were employed that recognize these proteins dually phosphorylated states. Nef treatment of cells induced a strong increase in phosphorylation of p3 8 above basal levels (FIG. 7C). In contrast, there was no detectable increase in the phosphorylation of p38 in Nef treated cells in the presence of Cpd4 following a 15-min exposure to the drug at a 1 μM dose. Parallel blots were run and probed with antibodies that detected total levels of p38. Exposure to Cpd4 did not alter the total levels of p38, which also confirmed equal protein loading. The data support that Nef can induce phosphorylation of the p38 MAP kinase an suggests that it is a direct activator of this pathway.

Further studies investigated whether Nef stimulated phosphorylation of c-Jun as well. Experiments were performed using an antibody that specifically recognizes phosphorylation-state independent levels of endogenous c-Jun protein. Exposure to Nef for 15 min resulted in strong phosphorylated c-Jun in the nuclear extracts of cells in a dose dependent manner. In contrast only very low levels of non phosphorylated c-Jun was observed in the non-stimulated controls. Two background bands were observed in the Nef treated group following treatment of cells with 1 μM Cpd4. These bands probably correspond to phospho c-Jun (the higher band) and non-phosphorylated form (lower band), which are known to be detected by this antibody. The blocking of Nef mediated phosphorylated of c-Jun in Cpd4 treated cells suggests that the levels of JNK observed does result in the transduction of signals through to c-Jun (FIG. 7D). The Cpd4 treatment was accompanied by a decrease in the phosphorylated status of c-Jun, a member of the AP-1 enhancer binding transcriptional activators.

### Discussion

The hallnark of HIV-1 infection typically involves the destruction of CD4⁺ T-cells and the suppression of cellular immune responses in vivo). Ironically, recent evidence suggests that effective immune responses and control of virema is heavily dependent on the generation of potent cellular responses. In fact, virus-specific CTLs attribute significantly to the control of acute phase infections and denote a mechanism by which viral loads can be controlled. Furthermore, studies on SIV infection models have also indicated that viremia and viral load could be effectively controlled through CD8⁺ T cells. In view of these findings, there have been some conflicting reports on the actual mechanism that HIV implements to induce the depletion of T-cells. For instance, two different mechanisms have been proposed for elimination of T cells during HIV infection. Clearance can be via direct apoptosis of infected cells or secondarily through bystander cell death. These two together would be expected to be significantly more potent in destroying immune function than either individually as direct infection and killing is likely restricted to CD4⁺ T cells while bystander killing is thought to involve CD8⁺ effector T cells, the cell populationrdirectly responsible for immune clearance. Indirect cell killing has been proposed to involve the upregulation of FasL to induce apoptosis of effector CTLs as they approach viral harboring CD4⁺ T cells. and macrophages. Hence this effect represents a significant advantage for HIV in evading immune recognition.

Recently it has been reported that these HIV infected cells are resistant to CTL induced killing and that his effect was dependent on the expression of the HIV-1 accessory gene nef Several mechanisms have been preliminarily proposed for these Nef mediated effects. This effect may involve the inhibition of ASK-1 mediated JNK signaling or through the indirect phosphorylation of Bad by PI-3-kinase and PAK. For example a recent report by Xu et al. (J Exp Med. 189(9):1489-96 (1999) which is incorporated herein by reference) reported that Nef binds to the ξ chain of the TCR and this interaction was important for the induction of FasL expression. In this study the induction of FasL expression was dependent on the proline rich domain (PxxP) domain located within the amino acids 73-82, which is known to interact with SH3 domains of protein tyrosine kinases. Hence, T-cell activation induction by Nef through the ξ chain appears to require the PxxP domain. Additionally, this domain was also crucial for FasL transcription by Nef, rendering the downstream signaling induced by the PxxP domain via Nef as a crucial regulator of bystander T-cell apoptosis. Accordingly, evidence suggests that Nef also coprecipitates with the Nef-associated kinase NAK), which is a member of the p21 related kinase family (PAK) and is activated via the small GTJases CDC42 and Rac1 through Vav. Moreover, Pak1 and 2 was implicated in this activation. Although it is believed that activation of FasL transcription finctions through the TCR-CD3⁺ complex mediated by the CD3⁺ ξ chain. None of these studies clearly elucidated to role of Nef in non T cell induced apoptosis of CD8⁺ effector T cells. In APC induced bystander apoptosis Nef has been implicated but what role any of the above play in signaling in these cells is currently unknown. In addition the essential transcription signals that Nef manifests to induce these apoptotic effects remain unknown. It is evident that clarification of such mechanisms is likely important. Overall the conclusion that HIV mediates immune escape in part through Nef by inducing apoptosis of bystander cells, while protecting itself against CTL induced death is likely to be very important in understanding HIV pathogenesis.

The results presented here indicate that a crucial downstream linkage of HIV mediated T-cell activation and FasL transcription is through the mitogen activated protein kinase p38/JNK pathway. Accordingly, treatment of HIV infected PBMCs with selective inhibitors of p38 MAPK severely attenuated or eliminated viral induced apoptosis FIG. 1A and FIG. 1B). This effect was not dade specific, as p38 blockade effectively abated cell death induced by multiple viral subtypes (FIG. 1C). Correspondingly, treatment of both Jurkat t cells and PBMCs with the potent p38 inhibitor (Cpd4) severely repressed the surface expression of FasL, indicating that HIV directs FasL-mediated bystander apoptosis through the p38 MAPK pathway.

Previous work by Xu et al. suggests that HIV mediated FasL induction is a direct consequence of Nef-induced functions. Our results reinforce this previous study, as significant FasL expression was only observed among Jurkat cells transfected with pNef, as compared to pVpr, pTat, and pEnv (FIG. 3A). More importantly, inhibition of p38 MAPK in these cells with Cpd4 thoroughly repressed Nef-induced FasL expression. In addition, this effect was extended into a viral infection setting, as p38 MAPK inhibition of viral infected PBMCs did not go on to express FasL. Therefore, p38 MAPK represents a crucial signal that Nef targets to induce FasL-mediated bystander apoptosis in the viral infection setting.

Nef induction of FasL has been suggested to play a role in bystander cell apoptosis. The ability of p38 MAPK inhibition to prevent bystander killing was analyzed. Previous studies indicate that HIV infection of macrophages induces FasL expression and drives significant bystander killing of CD4⁺ T-cells. Additionally, macrophages also drive FasL upregulation to induce bystander killing of HIV specific CD8⁺ T cells. The destruction of the CD8⁺ T cells by the FasL pathway is likely a significant damper on the cell-mediated immune response, which ultimately could limit immune clearance. Therefore, the ability of the p38 inhibition to modulate FasL expression and hence bystander killing between macrophages and CD8⁺ T-cells was also investigated. Co-culturing of HIV infected macrophages with autologous CD8⁺ T-cells with and without Cpd4 revealed that inhibition of p38 MAPK effectively blocked bystander apoptosis of CD8⁺ T-cells. In addition, this effect was also observed among PMBCs from HIV-1 infected patients when mixed with uninfected cells. Therefore, HIV-1 FasL-mediated bystander apoptosis is effectively blocked by p38/JNK inhibition within multiple settings.

Previous work suggests that multiple factors may regulate FasL expression including NFAT, c-Myc, NF-κB, and AP-1. Accordingly, the essential dependence of these factors in the context of Nef induced FasL expression context was uncertain. To study in the detail the biochemical mechanisms of this action, we transfected cells with a reporter vector that possesses AP-1 or NF-κB binding sites to drive luciferase transcription. In these studies, Nef was efficient at inducing the transcriptional activation of both AP-1 and NF-κB. More importantly, the p38 MAPK inhibitor effectively attenuated transcription of both transcriptional factors. In addition, the inhibitor was equally effective at decreasing phosphorylation mediated activation of p38 and hence resulting in a significant reduction of the phosphorylation of c-Jun. Therefore, the blockage of p38 by the inhibitor diminishes both signals, in part through decreased phosphorylation. More notably, based on prior findings on the dependence of NF-κB and AP-1 signals to mediate FasL induction, it is likely that the blockage of p38 represses its downstream transcriptional proteins and hence leads to the repression of FasL expression.

FasL induced bystander apoptosis is an essential immune evasive maneuver employed by HIV to avoid host detection by CTLs. Until now, the signaling mechanism that mediates this process remained elusive. The results herein indicate that p38 MAPK signaling is crucial for transcriptional activation of AP-1, which drives FasL induction by Nef within the viral setting. The kinase(s) that mediates Nef activation of p38 MAPK induction remains to be determined, but nonetheless this pathway represents a vital target for therapeutic development against HIV mediated T-cell depletion. As these therapeutics apparently target a host cell pathway of viral dependence, it is not unlikely that HIV would exhibit significant restriction in trying to circumvent this central host pathway. Further study of this pathway has importance for the development of novel therapeutics or anti viral combinations that could impact HIV pathogenesis.

### REFERENCES (WHICH ARE EACH INCORPORATED HEREIN BY REFERENCE)

Cohen J. Ground Zero: AIDS Research in Africa. Science 2000, 288:2150-2153.

Morison L. The global epidemilogy of HIV/AIDS. British Medical Bulletin. 58:7-18 (2001).

UNAIDS. AIDS epidemic update: December 2000. Geneva UNAIDS/WHO.

Letvin, N. L. Progress in the development of an HIV-1 vaccine. Science 280(5371), 1875-1880 (1998).

Temesgen Z. Current status of antiretroviral therapies. Expert Opin Pharmacother. 2(8):1239-1246. (2001).

Ho DD et al. Rapid turnover of plasma virions and CD4 lymphocytes in HIV-1 infection. Nature. 373: 123-126. (1995).

McMichael A. T cell responses and viral escape. Cell. 93:673-6. (1998).

Xu X N, Screaton G R, McMichael A J. Virus infections: escape, resistance, and counterattack. Immunity. 15(6):867-70 (2001).

Cullen B R. AIDS. The positive effect of the negative factor. Nature 351(6329):698-9 (1991).

Fisher A G, Ensoli B, Ivanoff L, Chamberlain M, Petteway S, Ratner L, Gallo R C, Wong-Staal F. The sor gene of HIV-1 is required for efficient virus transmission in vitro. Science 237(4817):888-93 (1987).

Colombini S, Arya S K, Reitz M S, Jagodzinski L, Beaver B, Wong-Staal F. Structure of simian immunodeficiency virus regulatory genes. Proc Natl Acad Sci U S A. 86(13):4813-7 (1989).

Frankel A D, Young J A. HIV-1: fifteen proteins and an RNA. Annu Rev Biochem; 67:1-25 (1998).

McCune J M. The dynamics of CD4+ T-cell depletion in HIV disease. Nature. 410(6831):974-9 (2001).

Finkel T H, Banda N K. 1994. Indirect mechanisms of HIV pathogenesis: how does HIV kill T cells? Curr. Opin. Immunol. 6(4):605-15.

D R Green & H M Beere Apoptosis: Mostly dead. Nature 412, 133-135 (2001).

Sieg S, Smith D, Yildirim Z, Kaplan D. Fas ligand deficiency in HIV disease. Proc Natl Acad Sci USA. 94(11):5860-5 (1997).

Mueller Y M, De Rosa S C, Hutton J A, Witek J, Roederer M, Altman J D, Katsikis P D. Increased CD95/Fas-induced apoptosis of HIV-specific CD8(+) T cells. Immunity. 15(6):871-82 (2001).

Tateyama M, Oyaizu N, McCloskey T W, Than S, Pahwa S. CD4 T lymphocytes are primed to express Fas ligand by CD4 cross-inking and to contribute to CD8 T-cell apoptosis via Fas/FasL death signaling pathway. Blood 96(1):195-202 (2000).

Gandhi R T, Chen B K, Straus S E, Dale J K, Lenardo M J, Baltimore D. 1998. HIV-1 directly kills CD4+ T cells by a Fas-independent mechanism. J. Exp. Med. 187:1113-22.

Ogg G. S., Jin X., Bonhoeffer S., Dunbar P. R., Nowak M. A., Monard S., Segal J. P., Cao Y., Rowland-Jones S. L., Cerundolo V., Hurley A., Markowitz M., Ho D. D., Nixon D. F., and McMichael A. J. 1998. Quantitation of HIV-1-specific cytotoxic T lymphocytes and plasma load of viral RNA. Science. 279:2103-6.

Finkel T H, Tudor-Williams G, Banda N K, Cotton M F, Curiel T, Monks C, Baba T W, Ruprecht R M, Kupfer A. 1995. Apoptosis occurs predominantly in bystander cells and not in productively infected cells of HIV- and SIV-infected lymph nodes. Nat. Med. 1:129-34.

Muro-Cacho C A, Pantaleo G, Fauci A S. 1995. Analysis of apoptosis in lymph nodes of HIV-infected persons. Intensity of apoptosis correlates with the general state of activation of the lymphoid tissue and not with stage of disease or viral burden. J. Immunol. 154:5555-66.

Westendorp M O, Frank R, Ochsenbauer C, Stricker K, Dhein J, Walczak H, Debatin K M, Krammer P H. Sensitization of T cells to CD95-mediated apoptosis by HIV-1 Tat and gp120. Nature. 375(6531):497-500 (1995).

Fackler O T, Baur A S. Live and let die: Nef functions beyond HIV replication. Immunity 16(4):493-7 (2002).

Ameisen J C. Apoptosis subversion: HIV-Nef provides both armor and sword. Nat Med. 7(11):1181-2 (2001).

Cuenda A, Rouse J, Doza Y N, Meier R, Cohen P, Gallagher T F, Young P R, Lee J C. SB 203580 is a specific inhibitor of a MAP kinase homologue which is stimulated by cellular stresses and interleulin-1. FEBS Lett. 364(2):229-33 (1995).

Wadsworth S A, Cavender D E, Beers S A, Lalan P, Schafer P H, Malloy E A, Wu W, Falmy B, Olini G C, Davis J E, Peliegrino-Gensey J L, Wachter M P, Siekierka JJ 1999. RWJ 67657, a potent, orally active inhibitor of p38 mitogen-activated protein kinase. Pharmacol Exp Ther. 291(2):680-687.

Muthumani K, Wadsworth S, Dayes N. S, Hwang D. S, Choo A. Y, Abeysinghe H. R, Siekierka J and Weiner D B. Suppression of HIV-1 viral replication and cellular pathogenesis by a novel p38/JNK kinase inhibitor (Submitted) 2003.

He, J., Choe, S., Walker, R., Di, Marzio, P., Morgan, D. O., and Landau, N. R. J. Virol. 69, 6705-6711 (1995).

Muthumani, K., Hwang, D. S., Desai, B M., Zhang, D Dayes, N., Green, D R and Weiner, D. B. HIV-1 Vpr Induces Apoptosis Through Caspase 9 in T Cells and PBMC's. Journal of Biol. Chem. 277: 37820-37831 (2002).

Fauci A S. Resistance to HIV-1 infection: it's in the genes. Nat Med. 2(9):966-967 (1996)

Vignaux F, Vivier E, Malissen B, Depraetere V, Nagata S, Golstein P. 1995. TCR/CD3 coupling to Fas-based cytotoxicity. J. Exp. Med. 181:781-6.

Latinis, K. M., L. L. Carr, E. J. Peterson, L. A. Norian, S. L. Eliason, and G. A. Koretzky. 1997. Regulation of CD95 (Fas) ligand expression by TCR-mediated signaling events. J. Immunol. 158: 4602-4611.

Xu X N, Laffert B, Screaton G R, Kraft M, Wolf D, Kolanus W, Mongkolsapay J, McMichael A J, Baur A S. Induction of Fas ligand expression by HIV involves the interaction of Nef with the T cell receptor zeta chain. J Exp Med. 189(9):1489-96 (1999).

Geleziunas R, Xu W, Takeda K, Ichijo H, Greene W C. HIV-1 Nef inhibits ASK1-dependent death signalling providing a potential mechanism for protecting the infected host cell. Nature. 410(6830):834-8 (2001).

Ahmad N, Venkatesan S. Nef protein of HIV-1 is a transcriptional repressor of HIV-1 LTR. Science. 241(4872):1481-5 (1988).

Collins K L, Chen B K, Kalams S A, Walker B D, Baltimore D. 1998. HIV-1 Nef protein protects infected primary cells against killing by cytotoxic T lymphocytes. Nature. 391:397-401.

Baur A S, Sawai E T, Dazin P, Fantl W J, Cheng-Mayer C, Peterlin B M. HIV-1 Nef leads to inhibition or activation of T cells depending on its intracellular localization. Immunity. 1(5):373-84 (1994).

Sawai E T, Khan I H, Montbriand P M, Peterlin B M, Cheng-Mayer C, and Luciw P A. 1996. Activation of PAK by HIV and SIV Nef: importance for AIDS in rhesus macaques. Curr. Biol. 6:1519-27.

Zauli G, Gibellini D, Secchiero P, Dutartre H, Olive D, Capitani S, Collette Y. Human immunodeficiency virus type 1 Nef protein sensitizes CD4(+) T lymphoid cells to apoptosis via functional upregulation of the CD95/CD95 ligand pathway. Blood 93(3):1000-10 (1999).

Rasola A, Gramaglia D, Boccaccio C, Comoglio P M. Apoptosis enhancement by the HIV-1 Nef protein. J Immunol. 1;166(1):81-8 (2001).

Borrow P., Lewicki H., Hahn B. H., Shaw G. M., and Oldstone M. B. 1994. Virus-specific CD8+ cytotoxic T-lymphocyte activity associated with control of viremia in primary human inunundeficiency virus type 1 infection. Journal of Virology. 68:6103-10.

Schmitz J. E., Kuroda M. J., Santra S., Sasseville V. G., Simon M. A., Lifton M. A., Racz P., Tenner-Racz K., Dalesandro M., Scallon B. J., Ghrayeb J., Forman M. A., Montefiori D. C., Rieber E. P., Letvin N. L., and Reimanm K. A. 1999. Control of viremia in simian immunodeficiency virus infection by CD8+ lymphocytes. Science. 283:857-60.

Barker E, Bossart K N, Fujimura S H, Levy J A. CD28 costimulation increases CD8+ cell suppression of HIV replication. J Immunol 1997 Nov. 15; 159(10):5123-31

Ventura J J, Kennedy N J, Lamb J A, Flavell R A, Davis R J. c-Jun NH(2)-terminal kinase is essential for the regulation of AP-1 by tumor necrosis factor. Mol Cell Biol 2003 23(8):2871-82

Dong C, Davis R J, Flavell R A. MAP kinases in the immune response. Annu Rev Immunol 2002;20:55-72

Kasibhatla S, Brunner T, Genestier L, Echeverri F, Mahboubi A, Green D R. 1998. DNA damaging agents induce expression of Fas ligand and subsequent apoptosis in T lymphocytes via the activation-of NF-kappa B and AP-1. Mol. Cell. 1:543-51.

Le-Niculescu H, Bonfoco E, Kasuya Y, Claret F X, Green D R, Karin M. 1999. Withdrawal of survival factors results in activation of the JNK pathway in neuronal cells leading to Fas ligand induction and cell death. Mol. Cell Biol. 19:751-63.

Kasibhatla S, Genestier L, Green D R. 1999. Regulation of fas-ligand expression during activation-induced cell death in T lymphocytes via nuclear factor kappaB. J. Biol. Chem. 274:987-92.

Brunner T, Kasibhatla S, Pinkoski M J, Frutschi C, Yoo N J, Echeverri F, Mahboubi A, Green D R. 2000. Expression of Fas ligand in activated T cells is regulated by c-Myc. J. Biol. Chem. 275:9767-72.

Faris, M., N. Kokot, K Latinis, S. Kasibhatla, D. R. Green, G. A. Koretzky, and A. Nel. 1998. The c-Jun N-terminal kinase cascade plays a role in stress-induced apoptosis in Jurkat cells by up-regulating Fas ligand expression. J. Immunol. 160: 134-144.

Lee F S, Hagler J, Chen Z J, Maniatis T.Activation of the IkappaB alpha kinase complex by MEKK1, a kinase of the JNK pathway. Cell 1997 Jan. 24;88(2):213-22

Tang G, Minemoto Y, Dibling B, Purcell N H, Li Z, Karin M, Lin A. Inhibition of JNK activation through NF-kappaB target genes. Nature, 414(6861):313-7 (2001).

Letvin N L, Barouch D H, Montefiori D C. Prospects for vaccine protection against HIV-1 infection and AIDS. Annu Rev Immunol. 20:73-99.(2002)

Barouch D H, Kunstrnan J, Kuroda M J, Schmitz J E, Santra S, Peyerl F W, Krivulka G R, Beaudry K, Lifton M A, Gorgone D A, Montefiori D C, Lewis M G, Wolinsky S M, Letvin N L. Eventual AIDS vaccine failure in a rhesus monkey by viral escape from cytotoxic T lymphocytes. Nature 415(6869):335-9 (2002).

Silvestri G, Sodora D L, Koup R A, Paiardini M, O'Neil S P, McClure H M, Staprans S I, Feinberg M B. Nonpathogenic SIV infection of sooty mangabeys is characterized by limited bystander immunopathology despite chronic high-level viremia. Immunity. 18(3):441-52 (2003).

Altfeld M, Allen T M, Yu X G, Johnston M N, Agrawal D, Korber B T, Montefiori D C, O'Connor D H, Davis B T, Lee P K, Maier E L, Harlow J, Goulder P J, Brander C, Rosenberg E S, Walker B D. HIV-1 superinfection despite broad CD8+ T-cell responses containing replication of the primary virus. Nature 420(6914):434-9 (2002).

Bobbitt K R, Addo M M, Altfeld M, Filzen T, Onafuwa A A, Walker B D, Collins K L. Rev activity determines sensitivity of HIV-1-infected primary T cells to CTL killing. Immunity. 18(2):289-99 (2003)

Wolf D, Witte V, Laffert B, Blume K, Stromer E, Trapp S, d'Aloja P, Schurmann A, Baur A S. HIV-1 Nef associated PAK and PI3-kinases stimulate Akt-independent Bad-phosphorylafion to induce anti-apoptotic signals. Nat Med (11):1217-24 (2001).

Saksela K, Cheng G, Baltimore D. Proline-rich (PxxP) motifs in HIV-1 Nef bind to SH3 domains of a subset of Src kinases and are required for the enhanced growth of Nef+ viruses but not for down-regulation of CD4. EMBO J. 14:484-91. (1995).

Nunn M F, Marsh J W. Human immunodeficiency virus type 1 Nef associates with a member of the p21-activated kinase family. J. of Virology. 70:6157-61 (1996).

Renkema, G. H., A. Manninen, D. A. Mann, M. Harris, and K. Saksela. 1999. Identification of the Nef-associated kinase as p21-activated kinase 2. Curr. Biol. 9:1407-1410.

Fackler O T, Luo W, Geyer M, Alberts A S, Peterlin B M. 1999. Activation of Vav by Nef induces cytoskeletal rearrangements and downstream effector functions. Mol. Cell. 3:729-39 (1999).

Fackler O T, Lu X, Frost J A, Geyer M, Jiang B, Luo W, Abo A, Alberts A S, Peterlin B M. p21-activated kinase 1 plays a critical role in cellular activation by Nef. Mol. Cell Biol. 20:2619-27 (2000).

Kaech S M, Hemby S, Kersh E, Ahmed R Molecular and functional profiling of memory CD8 T cell differentiation. Cell 111(6):837-51 (2002).

Wong P, Pamer E G. Feedback regulation of pathogen-specific T cell priming. Immunity. 18(4):499-511 (2003).

### Example 2

In some embodiments, the compounds have the structure (Formula 1) described in U.S. Pat. No. 5,965,583: 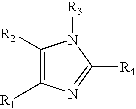
or a pharmaceutically acceptable salt thereof,
wherein:

R₁ is phenyl, substituted phenyl (where the substituents are selected from the group consisting of C₁₋₅ alkyl, halogen, nitro, trifluoromethyl, and nitrile), or heteroaryl where the heteroaryl contains 5 to 6 ring atoms;

R₂ is phenyl, substituted phenyl (where the substituents are selected from the group consisting of C₁₋₅ alkyl, halogen, nitro, trifluoromethyl, and nitrile), heteroaryl where the heteroaryl contains 5 to 6 ring atoms and is optionally C₁₋₄ alkyl substituted;

R₃ is hydrogen, SEM, C₁₋₅ alkoxycarbonyl, aryloxycarbonyl, aryl C₁₋₅ alkyloxycarbonyl, arylC₁₋₅ alkyl, substituted arylC₁₋₅ alkyl (where the aryl substituents are independently selected from one or more members of the group consisting of C₁₋₅ alkyl, C₁₋₅ alkoxy, halogen, amino, C₁₋₅ alkylamino, and diC₁₋₅ alkylamino), phthalimido C₁₋₅ alkyl, amino C₁₋₅ alkyl, diamino C₁₋₅ alkyl, succinimido C₁₋₅ alkyl, C₁₋₅ alkylcarbonyl, arylcarbonyl, C₁₋₅ alkylcarbonyl C₁₋₅ alkyl, aryloxycarbonyl C₁₋₅ alkyl, heteroaryl C₁₋₅ alkyl where the heteroaryl contains 5 to 6 ring atoms;

R₄ is -(A)-(CH₂)_{q}—X where A is vinylene, ethynylene or 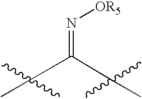
where R₅ is selected from the group consisting of hydrogen, C₁₋₅ alkyl, phenyl and phenyl C₁₋₅ alkyl;

q is 0-9;

X is selected from the group consisting of hydrogen, hydroxy, vinyl, substituted vinyl (where one or more substituents are selected from the group consisting of fluorine, bromine, chlorine and iodine), ethynyl, substituted ethynyl (where the substituents are selected from one or more of the group consisting of fluorine, bromine chlorine and iodine), C₁₋₅-alkyl, substituted C₁₋₅ alkyl (where the alkyl substituents are selected from the group consisting of one or more C₁₋₅ alkoxy trihaloalkyl, phthalimido and amino), C₃₋₇ cycloalkyl, C₁₋₅ alkoxy, substituted C₁₋₅ alkoxy (where the alkyl substituents are selected from the group consisting of phthalimido and amino), phthalimidooxy, phenoxy, substituted phenoxy (where the phenyl substituents are selected from the group consisting of C₁₋₅ alkyl, halogen and C₁₋₅ alkoxy), phenyl, substituted phenyl (where the phenyl substituents are selected from the group consisting of C₁₋₅ alkyl, halogen and C₁₋₅ alkoxy), aryl C₁₋₅ alkyl, substituted aryl C₁₋₅ alkyl (where the aryl substituents are selected from the group consisting of C₁₋₅ alkyl, halogen and C₁₋₅ alkoxy), aryloxy C₁₋₅ alkyl amino, C₁₋₅ alkylamino, di C₁₋₅ alkylamino, nitrile, oxime, benxyloxyimino, C₁₋₅ alkyloxyimino, phthalimido, succinimido, C₁₋₅ alkylcarbonyloxy, phenylcarbonyloxy, substituted phenylcarbonyoxy (where the phenyl substitutents are selected from the group consisting of C₁₋₅ alkyl, halogen and C₁₋₅ alkoxy), phenyl C₁₋₅ alkylcarbonyloxy, (where the phenyl substitutents are selected from the group consisting of C₁₋₅ alkyl, halogen and C₁₋₅ alkoxy), aminocarbonyloxy, C₁₋₅ alkylaminocarbonyloxy, diC₁₋₅ alkylaminocarbonyloxy, C₁₋₅ alkoxycarbonyloxy, substituted C₁₋₅ alkoxycarbonyloxy (where the alkyl substituents are selected from the group consisting of methyl, ethyl, isopropyl and hexyl), phenoxycarbonyloxy, substituted phenoxycarbonyloxy (where the phenyl substituents are selected from the group consisting of C₁₋₅ alkyl, C₁₋₅ alkoxy, and halogen), C₁₋₅ alkylthio, substituted C₁₋₅ alkylthio (where the alkyl substituents are selected from the group consisting of hydroxy and phthalimido), C₁₋₅ alkylsulfonyl, phenylsulfonyl, substituted phenylsulfonyl (where the phenyl substituents are selected from the group consisting of bromine, fluorine, chloride, C₁₋₅ alkoxy and trifluoromethyl); with the proviso:

if A is 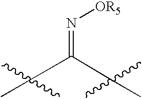
q is 0 and X is H, R₃ may not be SEM;
and pharmaceutically acceptable salts thereof.

Specific compounds include: 4-(4-fluorophenyl)-2-(4-hydroxybutyn-1-yl)-1-(3-phenylpropyl)-5-(4-pyridinyl)imidazole, 4-(4-fluorophenyl)-2-(3-hydroxypropyn-1-yl)-1-(3-phenylpropyl)-5-(4-pyridinyl)imidazole, 4(4-fluorophenyl)-2-(5-hydroxypentyn-1-yl)-1-(3-phenylpropyl)-5-(4-pyridinyl)imidazole, and 4-(4-fluorophenyl)-2-(6-hydioxyhexyn-1-yl)-1-(3-phenylptopyl)-5-(4-pyridin yl)imidazole, 4-(4-fluorophenyl)-2-(4-hydroxybutyn-1-yl)-1-(3-phenylpropyl)-5-(4-pyridin yl)imidazole, 4-(4-fluorophenyl)-2-(5-cyanopentyn-1-yl)-1-(3-phenylpropyl)-5-(4-pyridinyl)imidazole, 4-(4-fluorophenyl)-2-(4-dimethylaminobutyn-1-yl)-1-(3-phenylpropyl)-5-(4-pyridinyl)imidazole, 4-(4-fluorophenyl)-2-(4-(phenylcarbonyloxy)butyn-1-yl)-1-(3-phenylpropyl)-5-(4-pyridinyl)imidazole, 4-(4-fluorophenyl)-2-(4-methylcarbonyloxy)butyn-1-yl)-1-(3-phenylpropyl)-5-(4-pyridinyl)imidazole, 4-(4-fluorophenyl)-2-(3-cyclopentylpropyn-1-yl)-1-(3-phenylpropyl)-5-(4-pyridinyl)imidazole, and 4-(4-fluorophenyl)-2-(5-(butylsulfonyl)pentyn-1-yl)-1-(3-phenylpropyl)-5-(4-pyridinyl)imidazole, 4-4-fluorophenyl)-2-(octyn-1-yl)-1-(3-phenylpropyl)-5-(4-pyridinyl)imidazole, 4-(4-fluorophenyl)-2-(5-butylthiopentyn-1-yl)-1-(3-phenylpropyl)-5-(4-pyridinyl)imidazole, 4-(4-fluorophenyl)-2-(5-phenylpentyn-1-yl)-1-(3-phenylpropyl)-5-(4-pyridinyl)imidazole, 4-(4-fluorophenyl-2-(5-chloropentyn-1-yl)-1-(3-phenylpropyl)-5-(4-pyridinyl)imidazole, 4-(4-fluorophenyl)-2-(5-hydroxypentyn-1-yl)-1-(3-phthalimidolpropyl)-5-(4-pyridinyl)imidazole, 4-(4-fluorophenyl-2-(pentyn-1-yl)-1-(3-phenylpropyl)-5-(4-pyridinyl)imidazole, and 4-(4-fluorophenyl)-2-(5-N-succinimidopentyn-1-yl)-1-(3-phenylpropyl)-5-(4-pyridinyl)imidazole, and pharmaceutical salts thereof.

### Example 3

In some embodiments, the compounds have the structure (Structure 2) described in U.S. Pat. No. 6,040,320: 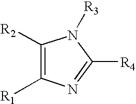
or a pharmaceutically acceptable salt thereof,
wherein:

R₁ is phenyl, heteroaryl wherein the heteroaryl contains 5 to 6 ring atoms, or substituted phenyl

wherein the substituents are independently selected from one or members of the group consisting of C₁₋₅ alkyl, halogen, nitro, trifluoromethyl and nitrile;

R₂ is phenyl, heteroaryl wherein the heteroaryl contains 5 to 6 ring atoms, substituted heteroaryl wherein the substituents are independently selected from one or more members of the group consisting of C₁₋₅ alkyl and halogen, or substituted phenyl wherein the substituents are independently selected from one or members of the group consisting of C₁₋₅ alkyl, halogen, nitro, trifluoromethyl and nitrile;

R₃ is hydrogen, SEM, C₁₋₅ alkoxycarbonyl, aryloxycarbonyl, arylC₁₋₅ alkyloxycarbonyl, arylC₁₋₅ alkyl, phthalimido₁₋₅ alkyl, aminoC₁₋₅ alkyl, diaminoC₁₋₅ alkyl, succinimidoC₁₋₅ alkyl, C₁₋₅ alkylcarbonyl, arylcarbonyl, C₁₋₅ alkylcarbonylC₁₋₅ alkyl, aryloxycarbonylC₁₋₅ alkyl, heteroarylC₁₋₅ alkyl where the heteroaryl contains 5 to 6 ring atoms, or substituted arylC₁₋₅ alkyl wherein the aryl substituents are independently selected from one or more members of the group consisting of C₁₋₅ alkyl, C₁₋₅ alkoxy, halogen, amino, C₁₋₅ alkylamino, and diC₁₋₅ alkylamino;

R₄ is (A)ₙ-(CH₂)_{q}—X wherein:
A is sulfur or carbonyl;
n is 0 or 1;
q is 0-9;

X is selected from the group consisting of hydrogen, hydroxy, halogen, vinyl, ethynyl, C₁₋₅ alkyl, C₃₋₇ cycloalkyl, C₁₋₅ alkoxy, phenoxy, phenyl, arylC₁₋₅ alkyl, amino, C₁₋₅ alkylamino, nitrile, phthalimido, amido, phenylcarbonyl, C₁₋₅ alkylaminocarbonyl, phenylaminocarbonyl, arylC₁₋₅ alkylaminocarbonyl, C₁₋₅ alkylthio, C₁₋₅ alkylsulfonyl, phenylsulfonyl, substituted sulfonamido wherein the sulfonyl, substituent is selected from the group consisting of C₁₋₅ alkyl, phenyl, araC₁₋₅ alkyl, thienyl, furanyl, and naphthyl; substituted vinyl wherein the substituents are independently selected from one or members of the group consisting of fluorine, bromine, chlorine and iodine, substituted ethynyl wherein the substituents are independently selected from one or more members of the group consisting of fluorine, bromine chlorine and iodine, substituted C₁₋₅ alkyl wherein the substituents are selected from the group consisting of one or more C₁₋₅ alkoxy, trihaloalkyl, phthalimido and amino, substituted phenyl wherein the phenyl substituents are independently selected from one or more members of the group consisting of C₁₋₅ alkyl, halogen and C₁₋₅ alkoxy, substituted phenoxy wherein the pheniyl substituents are independently selected from one or more members of the group consisting of C₁₋₅ alkyl, halogen and C₁₋₅ alkoxy, substituted C₁₋₅ alkoxy wherein the alkyl substituent is selected from the group consisting of phthalimido and amino, substituted arylC₁₋₅ alkyl wherein the alkyl substituent is hydroxyl, substituted arylC₁₋₅ alkyl wherein the phenyl substituents are independently selected from one or more members of the group consisting of C₁₋₅ alkyl, halogen and C₁₋₅ alkoxy, substituted amido wherein the carbonyl substituent is selected from the group consisting of C₁₋₅ alkyl, phenyl, arylC₁₋₅ alkyl, thienyl, furanyl, and naphthyl, substituted phenylcarbonyl wherein the phenyl substituents are independently selected from one or members of the group consisting of C₁₋₅ alkyl, halogen and C₁₋₅ alkoxy, substituted C₁₋₅ alkylthio wherein the alkyl substituent is selected from the group consisting of hydroxy and phthalimido, substituted C₁₋₅ alkylsulfonyl wherein the alkyl substituent is selected from the group consisting of hydroxy and phthalimido, substituted phenylsulfonyl wherein the phenyl substituents are independently selected from one or members of the group consisting of bromine, fluorine, chlorine, C₁₋₅ alkoxy and trifluoromethyl, with the proviso:

if A is sulfur and X is other than hydrogen, C₁₋₅ alkylaminocarbonyl, phenylaminocarbonyl, arylC₁₋₅ alkylaminocarbonyl, C₁₋₅ alkylsulfonyl or phenylsulfonyl, then q must be equal to or greater than 1;

if A is sulfir and q is 1, then X cannot be C₁₋₂ alkyl;

if A is carbonyl and q is 0, then X cannot be vinyl, ethynyl, C₁₋₅ alkylaminocarbonyl, phenylaminocarbonyl, arylC₁₋₅ alkylaminocarbonyl,C₁₋₅ alkylsulfonyl or phenylsulfonyl;

if A is carbonyl, q is 0 and X is H, then R₃ is not SEM;

if n is 0 and q is 0, then X cannot be hydrogen;

and pharmaceutically acceptable salts thereof.

Compounds include 5(4)-4-fluorophenyl)-2-(3-(naphth-1-ylamido)prop-1-yl)thio-4(5)-(4-pyridyl)-imidazole; and 5(4)-(4-fluorophenyl)-2-(3-(phenylsulfonamido)prop-1-yl)thio-4(5)-(4-pyridyl)-imidazole.

### Example 4

In some embodiments, the compounds have the structure (Structure 3) described in U.S. Pat. No. 6,147,096: 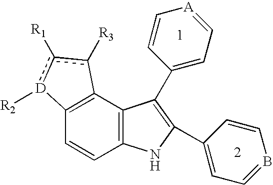
or a pharmaceutically acceptable salt thereof,
wherein:

(a) R₁, R₂ and R₃ are independently selected from the group consisting of (i) hydrogen, (ii) C₁₋₅ alkyl, (iii) C₁₋₅ alkylamino, (iv) diC₁₋₅ alkylamino, (v) a phenyl substituted with one or more of hydrogen, halogen, C₁₋₅ alkyl, and trihaloC₁₋₅ alkyl, and (vi) a phenylC1-5 alkyl substituted with one or more of hydrogen, halogen, C₁₋₅ alkyl, and trihaloC₁₋₅ alkyl;

(b) rings 1 and 2 are each independently substituted with one or more substituents selected from the group consisting of hydrogen, halogen, C₁₋₅ alkyl, and trihaloC₁₋₅ alkyl;

(c) A and B are independently nitrogen or carbon, at least one of A and B being nitrogen;

(d) D and E are nitrogen, with the proviso that (i) a double bond exists between the non-aryl carbon and either D or E, (ii) R₂ is absent if the double bond exists between the non-aryl carbon and D, and (iii) R₃ is absent if the double bond exists between the non-aryl carbon and E; and

(e) the compound is neither 1,6-dihydro-7-(4-pyridyl)-8-(4-fluorophenyl)-2-phenylmethyl-pyrrolo[3,2-e] benzimidazole, nor 3,6-dihydro-8-(4-fluorophenyl)-3-(3-phenylpropyl)-7-4-pyridyl)-pyrrolo[3,2-e]benzimidazole.

In some embodiments, the compound is selected from the group consisting of (i) 1,6-dihydro-7-(4-fluorophenyl)-8-(4-pyridyl)-2-phenyl-pyrrolo[3,2-e]benzimidazole; (ii) 1,6-dihydro-7-(4-fluorophenyl)-8-(4-pyridyl)-2-butyl-pyrrolo[3,2-e]benzimidazole; (iii) 1,6-dihydro-7-(4-fluorophenyl)-8-(4-pyridyl)-2-(2-phenylethyl)-pyrrolo[3,2-e]benzimidazole; (iv) 1,6-dihydro-7-(4-pyridyl)-8-(4-fluorophenyl)-pyrrolo[3,2-e]benzimidazole; and (v) 1,6-dihydro-7-(4-fluorophenyl)-8-(4-pyridyl)-2-phenylmethyl-pyrrolo[3,2-e]benzimidazole.

### Example 5

In some embodiments, the compounds have the structure (Structure 4) described in U.S. Pat. No. 6,214,830, 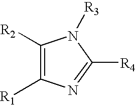
or a stereoisomer, solvate, or a pharmaceutically acceptable salt thereof, and a pharmaceutically acceptable carrier, diluent or excipient,
wherein:

R₁ is phenyl, substituted phenyl (where the substituents are selected from the group consisting of C1-5 alkyl, halogen, nitro, trifluoromethyl, and nitrile), or heteroaryl where the heteroaryl contains 5 to 6 ring atoms;

R₂ is phenyl, substituted phenyl (where the substituents are selected from the group consisting of C₁₋₅ alkyl, halogen, nitro, trifluoromethyl, and nitrile), heteroaryl where the heteroaryl contains 5 to 6 ring atoms and is optionally C₁₋₄ alkyl substituted;

R₃ is hydrogen, SEM, C₁₋₅ alkoxycarbonyl, aryloxycarbonyl, arylC₁₋₅ alkyloxycarbonyl, arylC₁₋₅ alkyl, substituted arylC₁₋₅ alkyl (where the aryl substituents are independently selected from one or more members of the group consisting of C₁₋₅ alkyl, C₁₋₅ alkoxy, halogen, amino, C₁₋₅ alkylamino, and diC₁₋₅ alkylamino), phthalimidoC₁₋₅ alkyl, aminoC₁₋₅ alkyl, diaminoC₁₋₅ alkyl, succinimidoC₁₋₅ alkyl, C₁₋₅ alkylcarbonyl, arylcarbonyl, C₁₋₅ alkylcarbonylC₁₋₅ alkyl, aryloxycarbonylC₁₋₅ alkyl, heteroarylC₁₋₅ alkyl where the heteroaryl contains 5 to 6 ring atoms;

R₄ is -(A)-CH₂)_{q}—X where:

A is vinylene, ethynylene or

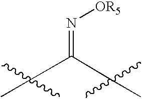

where

R₅ is selected from the group consisting of hydrogen, C₁₋₅ alkyl, phenyl and phenylC₁₋₅ alkyl;

q is 0-9;

X is selected from the group consisting of hydrogen, hydroxy, vinyl, substituted vinyl (where one or more substituents are selected from the group consisting of fluorine, bromine, chlorine and iodine), ethynyl, substituted ethynyl (where the substituents are selected from one or more of the group consisting of fluorine, bromine chlorine and iodine), C₁₋₅ alkyl, substituted C₁₋₅ alkyl (where the alkyl substituents are selected from the group consisting of one or more C₁₋₅ alkoxy trihaloalkyl, phthalimido and amino), C₃₋₇ cycloalkyl, C₁₋₅ alkoxy, substituted C₁₋₅ alkoxy (where the alkyl substituents are selected from the group consisting of phthalimido and amino), phthalimidooxy, phenoxy, substituted phenoxy (where the phenyl substituents are selected from the group consisting of C₁₋₅ alkyl, halogen and C₁₋₅ alkoxy), phenyl, substituted phenyl (where the phenyl substituents are selected from the group consisting of C₁₋₅ alkyl, halogen and C₁₋₅ alkoxy), arylC₁₋₅ alkyl, substituted arylC₁₋₅ alkyl (where the aryl substituents are selected from the group consisting of C₁₋₅ alkyl, halogen and C₁₋₅ alkoxy), aryloxyC₁₋₅ alkylamino, C₁₋₅ alkylamino, diC₁₋₅ alkylamino, nitrile, oxime, benxyloxyimino, C₁₋₅ alkyloxyimino, phthalimido, succinimido, C₁₋₅ alkylcarbonyloxy, phenylcarbonyloxy, substituted phenylcarbonyloxy (where the phenyl substitutents are selected from the group consisting of C₁₋₅ alkyl, halogen and C₁₋₅ alkoxy), phenylC₁₋₅ alkylcarbonyloxy, (where the phenyl substitutents are selected from the group consisting of C₁₋₅ alkyl, halogen and C₁₋₅ alkoxy), aminocarbonyloxy, C₁₋₅ alkylaminocarbonyloxy, diC₁₋₅ alkylaminocarbonyloxy, C₁₋₅ alkoxycarbonyloxy, substituted C₁₋₅ alkoxycarbonyloxy (where the alkyl substituents are selected from the group consisting of methyl, ethyl, isopropyl and hexyl), phenoxycarbonyloxy, substituted phenoxycarbonyloxy (where the phenyl substituents are selected from the group consisting of C₁₋₅ alkyl, C₁₋₅ alkoxy, and halogen), C₁₋₅ alkylthio, substituted C₁₋₅ alkylthio (where the alkyl substituents are selected from the group consisting of hydroxy and phthalimido), C₁₋₅ alkylsulfonyl, phenylsulfonyl, substituted phenylsulfonyl (where the phenyl substituents are selected from the group consisting of bromine, fluorine, chloride, C₁₋₅ alkoxy and trifluoromethyl); with the proviso:

if A is 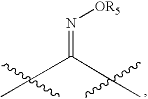
q is 0 and X is H, R3 may not be SEM;
and pharmaceutically acceptable salts thereof.

### Example 6

In some embodiments, the compounds have the structure (Structure 5) described in U.S. Pat. No. 6,469,174 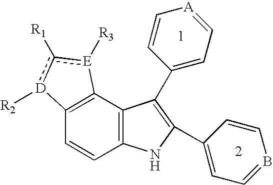
or a pharmaceutically acceptable salt thereof, wherein:

(a) R₁, R₂ and R₃ are independently selected from the group consisting of (i) hydrogen, (ii) C₁₋₅ salkyl, (iii) C₁₋₅ alkylamino, (iv) diC₁₋₅alkylamino, (v) a phenyl substituted with one or more of hydrogen, halogen, C₁₋₅ alkyl, and trihaloC₁₋₅ alkyl, and (vi) a phenylC₁₋₅ alkyl substituted with one or more of hydrogen, halogen, C₁₋₅ alkyl, and trihaloC₁₋₅ alkyl;

(b) rings 1 and 2 are each independently substituted with one or more substituents selected from the group consisting of hydrogen, halogen, C₁₋₅ alkyl, and trihaloC₁₋₅ alkyl;

(c) A and B are independently nitrogen or carbon, at least one of A and B being nitrogen;

(d) D and E are nitrogen, with the proviso that (i) a double bond exists between the non-aryl carbon and either D or E, (ii) R₂ is absent if the double bond exists between the non-aryl carbon and D, and (iii) R₃ is absent if the double bond exists between the non-aryl carbon and E; and

(e) the compound is neither 1,6-dihydro-7-(4-pyridyl)-8-(4-fluorophenyl)-2-phenylmethyl-pyrrolo[3,2-e]benzimidazole, nor 3,6-dihydro-8-(4-fluorophenyl)-3-(3-phenylpropyl)-7-(4-pyridyl)-pyrrolo[3,2-e]benzimidazole.

In some embodiments the compound is selected from the group consisting of: (i) 1,6-dihydro-7-(4-fluorophenyl)-8-(4-pyridyl)-2-phenyl-pyrrolo[3,2-e]benzimidazole; (ii) 1,6-dihydro-7-(4-fluorophenyl)-8-(4-pyridyl)-2-butyl-pyrrolo[3,2-e]benzimidazole; (iii) 1,6-dihydro-7-(4-fluorophenyl)-8-(4-pyridyl)-2-(2-phenylethyl)-pyrrolo[3,2-e]benzimidazole; (iv) 1,6-dihydro-7-(4-pyridyl)-8-(4-fluorophenyl)-pyrrolo[3,2-e]benzimidazole; and (v) 1,6-dihydro-7-(4-fluorophenyl)-8-(4-pyridyl)-2-phenylmethyl-pyrrolo[3,2-e]benzimidazole.

### Example 7

In some embodiments, the compounds have the structure (Structure 6) described in U.S. Pat. No. 6,410,540: 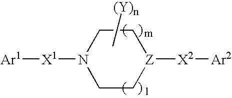
and the pharmaceutically acceptable salts thereof, or a pharmaceutical composition thereof,
wherein

Z is N or CR¹, R¹ is a noninterfering substituent,

each of X¹ and X² is a linker,

Ar¹ and Ar² are identical or different, and represent optionally substituted C₁-C₂₀ hydrocarbyl residues wherein at least one of Ar¹ and Ar² is an optionally substituted aryl group, with the proviso that when X² is CH₂ or an isostere thereof, X¹ is CO or an isostere thereof, and Ar² is optionally substituted phenyl, Ar¹ is other than an optionally substituted indolyl, benzimidazolyl or benzotriazolyl substituent, and wherein said optionally substituted phenyl is not an optionally substituted indolyl, benzimidazolyl, or benzotriazolyl,

Y is a noninterfering substituent, wherein n is an integer from 0-4, and

wherein m is an integer from 0-4 and 1 is an integer from 0-3.

Some compounds include: 1-(2-methoxy-4-hydroxybenzoyl)-4-benzylpiperidine; 1-(2-methoxy-4-methoxybenzoyl)-4-benzylpiperidine; 1-(2-methoxy-4-benzyloxybenzoyl)-4-benzylpiperidine; and 1-(2-methoxy-4-methoxybenzoyl)-4-(4-fluorobenzyl)piperidine.

### Example 8

In some embodiments, the compounds have the structure (Structure 7) described in U.S. Pat. No. 6,476,031: 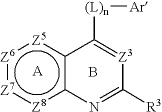
or the pharmaceutically acceptable salts thereof
wherein

R³ is a noninterfering substituent;

each Z is CR² or N, wherein no more than two Z positions in ring A are N, and wherein two adjacent Z positions in ring A cannot be N;

each R² is independently a noninterfering substituent;

L is a linker;

n is 0 or 1; and

Ar′ is the residue of a cyclic aliphatic, cyclic heteroaliphatic, aromatic or heteroaromatic moiety optionally substituted with 1-3 noninterfering substituents.

Some compounds are selected from the group consisting of: 2-phenyl4-(4-pyridylamino)-quinazoline; 2-(2-bromophenyl)-4-(4-pyridylamino)-quinazoline; 2-(2-chlorophenyl)-4-(4-pyridylamino)-quinazoline; 2-(2-fluorophenyl)-4-(4-pyridylamino)-quinazoline; 2-(2-methylphenyl)-4-(4-pyridylamino)-quinazoline; 2-(4-fluorophenyl)4-(4-pyridylamino)-quinazoline; 2-(3-methoxyaniyl)-4-(4-pyridylamino)-quinazoline; 2-(2,6-dichlorophenyl)-4-(4-pyridylamino)-quinazoline; 2-(2,6-dibrophonyl)-4-(4-pyridylamino)-quinazoline; 2-(2,6-difluomrophenyl)-4-(4-pyridylamino)-quinazoline; 2-(2-fluorophenyl)-4-(4-pyridylamino)-6,7-dimethoxyquinazoline; 2-(4-fluorophenyl)-4-(4-pyridylamino)-6,7-dimethoxyquinazoline; 2-(2-fluorophenyl)-4-(4-pyridylamino)-6-nitroquinazoline; 2-(2-fluorophenyl)-4-(4-pyridylamino)-6-aminoquinazoline; 2-(2-fluorophenyl)-4-(4-pyxdylamino)-7-aminoquinazoline; 2-(2-fluorophenyl)-4-(4-pyridylamino)-6-(3-methoxybenzylamino)-quinazoline; 2-(2-fluorophenyl)-4-(4-pyridylamino)-6-(4methoxybenzyrlamino)-quinazoiine; 2-(2-fluorophenyl)-4-(4-pyridylamino)-6(2-isobutylamino)-quinazoline; and 2-(2-fluorophenyl)-4-(4-pyridylamino)-6(4methymercaptobenzylamino)-quinazol ine.

### Example 9

In some embodiments, the compounds have the structure (Structure 8) described in U.S. Pat. No. 6,448,257 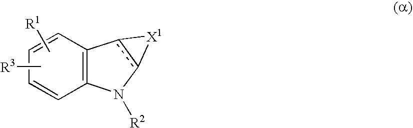
(wherein the dotted line represents an optional bond) preferably those of the formulas: 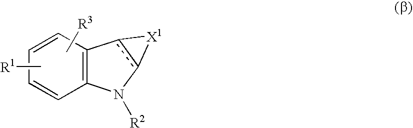
and the pharmaceutically acceptable salts thereof, wherein

X¹ is an alkyl bridge optionally containing an O, S, or N heteroatom that forms a fused aliphatic 5-7 membered ring and is optionally substituted by one or more of halo, OR, SR, NR_{2,} RCO, COOR, CONR2, OOCR, or NROCR where R is H or alkyl (1-6C), or by one or more CN or ═O, or by one or more aliphatic or aromatic 5- or 6-membered rings optionally containing 1-2 heteroatoms;

R¹ is 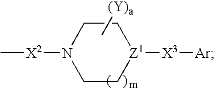
wherein

X² is CO or an isostere thereof;

m is 0 or 1;

Y is optionally substituted alkyl, optionally substituted aryl, or optionally substituted arylalkyl or two Y taken together may form an alkylene (2-3C) bridge;

n is 0-4;

Z¹ is CH or N;

X³ is CH or CHR where R is H or alkyl (1-6C), or an isostere thereof; and

Ar consists of one or two phenyl moieties directly coupled to X³ optionally substituted by halo, nitro, alkyl (1-6C), alkenyl (1-6C), alkynyl (1-6C), CN or CF3, or by RCO, COOR, CONR₂, NR_{2,} OR, SR, OOCR or NROCR wherein R is H or alkyl (1-6C) or by phenyl, itself optionally substituted by the foregoing substituents;

R² is H, or is alkyl (1-6C) or aryl each of said alkyl or aryl optionally including one or more heteroatoms which are O, S or N, and optionally substituted by one or more of halo, OR, SR, NR₂, RCO, COOR, CONR₂, OOCR, or NROCR where R is H or alkyl (1-6C), or by one or more CN or ═O, or by one or more aliphatic or aromatic 5- or 6-membered rings optionally containing 1-2 heteroatoms;

R³ is H, halo, NO₂, alkyl (1-6C), alkenyl (1-6C), alkynyl (1-6C), CN, OR, SR, NR₂, RCO, COOR, CONR₂, OOCR, or NROCR where R is H or alkyl (1-6C).

### Example 10

In some embodiments, the compounds have the structure (Structure 9) described in U.S. Pat. No. 6,479,507: 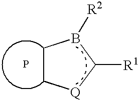
wherein:

R¹ is heteroaryl;

[symbol] represents a bond between either B and CR¹ or Q and CR¹ such that:

(i) when [symbol] is between Q and —CR¹— then:

B is nitrogen;

R² is aryl; and

Q is —CR— wherein:

R is hydrogen, alkyl, haloalkyl, cycloalkyl, cycloalkylalkyl, heteroalkyl, acyl, heterocyclyl, heterocyclylalkyl, heterocyclylcarbonyl, nitro, cyano, amino, monosubstituted amino, disubstituted amino, acylamino, sulfonylamino, —OR⁵ (where R⁵ is hydrogen, alkyl, heteroalkyl or heterocyclylalkyl), —COOR⁷ (where R⁷ is hydrogen or alkyl) or —CONR′R″ (where R′ and R″ independently represent hydrogen, alkyl or heteroalkyl); and

(ii) when [symbol] is between B and —CR1- then:

B is carbon;

R² is aryl or heteroaryl; and

Q is —NR4-, —O—, or —S— wherein:

R⁴ is hydrogen, alkyl, alkenyl, alkynyl, haloalkyl, cycloalkyl, cycloalkylalkyl, heteroalkyl, acyl, aralkyl, heteroaralkyl, heterocyclyl, heterocyclylalkyl, heterocyclylcarbonyl, —OR⁵ (where R⁵ is hydrogen, alkyl, heteroalkyl or heterocyclylalkyl), —SO₂R″ (where R″ is alkyl, amino, monosubstituted amino or disubstituted amino), —CONR′R″ (where R′ and R″ independently represent hydrogen, alkyl or heteroalkyl), -(alkylene)-Z or -(alkylene)-CO-(alkylene)-Z wherein:

Z is cyano; —COOR⁷ where R⁷ is hydrogen or alkyl; —CONR⁸R⁹ where R⁸ is hydrogen or alkyl, R⁹ is alkoxy or -(alkylene)-COOR⁷, or R⁸ and R⁹ together with the nitrogen atom to which they are attached form a heterocycle;

—C(═NR¹⁰)(NR¹¹R¹²) where R¹⁰, R¹¹ and R¹² independently represent hydrogen or alkyl, or R¹⁰ and R¹¹ together are —(CH₂)ₙ— where n is 2 or 3 and R12 is hydrogen or alkyl; or

—COR¹³ where R¹³ is alkyl, heteroalkyl, heterocyclylalkyl, aryl, aralkyl, heteroaryl or heteroaralkyl; and 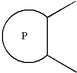
is a group represented by formula (S), (T), (U), (V) or (W); 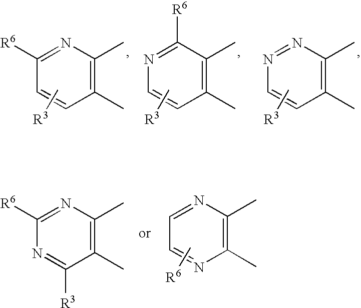
where:

R⁶ is hydrogen, alkyl, heteroalkyl, heterocyclylalkyl, halo, cyano, nitro, amino, monosubstituted amino, disubstituted amino, —COOR¹⁴, -(alkylene)-COOR¹⁴ (where R¹⁴ is hydrogen or alkyl), —CONR¹⁵ R¹⁶ (where R¹⁵ and R¹⁶ independently represent hydrogen or alkyl, or R¹⁵ and R¹⁶ together with the nitrogen atom to which they are attached form a heterocycle), —S(O)n R¹⁷ (where n is an integer from 0 to 2 and R¹⁷ is alkyl, amino, monosubstituted amino or disubstituted amino), —OR¹⁸ (where R¹⁸ is hydrogen, alkyl, heteroalkyl or heterocyclylalkyl), —NRC(O)R″ [where R is hydrogen, alkyl or: hydroxyalkyl and R″ is hydrogen, alkyl, cycloalkyl or -(alkylene)-X where X is hydroxy, alkoxy, amino, alkylamino, dialkylamino, heterocyclyl or

—S(O)n R′ (where n is 0 to 2 and R′ is alkyl)], —NRSO₂ R″ [where R is hydrogen or alkyl and R″ is alkyl or -(alkylene)-X where X is hydroxy, alkoxy, amino, alkylamino, dialkylamino or —S(O)ₙ R′ (where n is 0 to 2 and R′ is alkyl)]; and R³ is hydrogen, alkyl, alkenyl, alkynyl, haloalkyl, heteroalkyl, cycloalkyl, cycloalkylalkyl, cycloalkylthio, aralkyl, heteroaralkyl, heterocyclyl, heterocyclylalkyl, halo, cyano, nitro, amino, monosubstituted amino, disubstituted amino, acylamino, sulfonylamino, —OR¹⁹ (where R¹⁹ is hydrogen, alkyl, heteroalkyl or heterocyclylalkyl), —COOR²⁰ (where R²⁰ is hydrogen or alkyl), —CONR²¹ R²² (where R²¹ and R²² independently represent hydrogen, alkyl or heteroalkyl, or R²¹ and R²² together with the nitrogen atom to which they are attached form a heterocycle),

—S(O)n R²³ (where n is an integer from 0 to 2 and R²³ is alkyl, heteroalkyl, amino, monosubstituted amino or disubstituted amino), -(alkylene)-Z″ or -(alkylene)-CO-(alkylene)-Z″ wherein:

Z″ is cyano;

—COOR²⁴ where R²⁴ is hydrogen or alkyl;

—CONR²⁵ R²⁶ where R²⁵ and R²⁶ independently represent hydrogen or alkyl, or R²⁵ and R²⁶ together with the nitrogen atom to which they are attached form a heterocycle;

—C(═NR²⁷)(NR²⁸ R²⁹) where R²⁷, R²⁸ and R²⁹ independently represent hydrogen or alkyl, or R²⁷ and R²⁸ together are —(CH₂)ₙ— where n is 2 or 3 and R²⁹ is hydrogen or alkyl; or

—COR³⁰ where R³⁰ is alkyl, heteroalkyl, heterocyclylalkyl, aryl, aralkyl, heteroaryl or heteroaralkyl; and their pharmaceutically acceptable salts, prodrugs, individual isomers, and mixtures of isomers, provided that both R³ and R⁶ are not either amino, monosubstituted amino or disubstituted amino.
Alternatively, 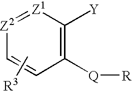
wherein:

One of Z¹ and Z² is nitrogen and the other is —CR⁶— wherein R⁶ is hydrogen, alkyl, or alkoxy; or both Z¹ and Z² are nitrogen such that:

(a) when Z¹ or Z² is nitrogen and the other is —CR⁶— then:

(i) when Y is halo, then -Q-R is —NH—C(R¹)═CH(R²) or —N═C(CH₃)(R¹);

(ii) when Y is —C(R²)═C(R¹)OX (where X is p-CH₃ C₆ H₄ SO₂—, CH₃ SO₂—, or CF₃ SO₂—), then -Q-R is nitro or amino; and

(iii) when Y is —C(O)R₂, then -Q-R is —NHC(O)R¹, —OCH₂ CO₂ R, or —SCH₂ R¹ where:

R is alkyl;

R¹ is heteroaryl; and

R² is aryl or heteroaryl; and

(b) when Z¹ and Z² both are nitrogen then:

Y is halo and -Q-R is —NH—C(R)═CH(R²) where R¹ and R² are as defined above; and

R³ is hydrogen, alkyl, halo, or alkoxy.
Alternatively 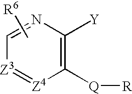
wherein:

Z³ is nitrogen and Z⁴ is —CR³— wherein R³ is hydrogen, alkyl, or alkoxy; or Z⁴ is nitrogen and Z³ is —CH—; such that:

(a) when Y is hydrogen, then -Q-R is —NH—N═C(R¹)CH₂ (R²); and

(b) when Y is —C(O)R², then -Q-R is —OCH₂ CO₂ R or —SCH₂ R¹ where:

R is alkyl;

R¹ is heteroaryl; and

R² is aryl or heteroaryl; and

R⁶ is hydrogen, alkyl, halo, or alkoxy.

Pharmaceutical compositions containing a therapeutically effective amount of compounds or pharmaceutically acceptable salts and a pharmaceutically acceptable excipient are also included.

### Example 11

In some embodiments, the compounds have the structure (Structure 10) described in U.S. Pat. No. 6,509,361 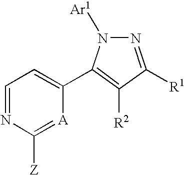
wherein

A is ═N— or ═CH—;

Ar¹ is an aryl group that is optionally substituted by one or more substituents selected from the group consisting of a halogen, hydrocarbyl, hydrocarbyloxy, nitro, cyano, perfluorohydrocarbyl, trifluoromethylhydrocarbyl, perfluorohydrocarbyloxy, hydroxy, mercapto, hydroxycarbonyl, aryloxy, arylthio, sulfonyl or sulfoxido, wherein the subsituent on the sulfur atom is hydrocarbyl, sulfonylamide,

wherein the substituents on the sulfonamido nitrogen atom are hydrido or hydrocarbyl, arylamino, arylhydrocarbyl, aryl, heteroaryloxy, heteroarylthio, heteroarylamino, heteroarylhydrocarbyl, hydrocarbyloxycarbonyl-hydrocarbyl, heterocyclooxy, hydroxycarbonyl-hydrocarbyl, heterocyclothio, heterocycloamino, cyclohydrocarbyloxy, cyclohydrocarbylthio, heteroarylhydrocarbyloxy, heteroarylhydrocarbylthio, heteroarylhydrocarbylamino, arylhydrocarbyloxy, arylhydrocarbylthio, arylhydrocarbylamino, heterocyclic, heteroaryl, hydroxycarbonylhydrocarbyloxy, hydrocarbyloxycarbonylhydrocarbyloxy, hydrocarbyloyl, arylcarbonyl, arylhydrocarbyloyl, hydrocarboyloxy, arylhydrocarboyloxy, hydroxyhydrocarbyl, hydroxyhydrocarbyloxy, hydrocarbylthio, hydrocarbyloxyhydrocarbylthio, hydrocarbyloxycarbonyl, hydroxycarbonylhydrocarbyloxy, hydrocarbyloxycarbonylhydrocarbyl, hydrocarbylhydroxycarbonylhydrocarbylthio, hydrocarbyloxycarbonylhydrocarbyloxy, hydrocarbyloxycarbonylhydrocarbylthio, hydrocarbylcarbonylamino, arylcarbonylamino, cyclohydrocarbylcarbonylamino, heterocyclohydrocarbylcarbonylamino, arylhydrocarbylcarbonylamino, heteroarylcarbonylamino, heteroarylhydrocarbylcarbonylamino, heterocyclohydrocarbyloxy, hydrocarbylsulfonylamino, arylsulfonylamino, arylhydrocarbylsulfonylamino, heteroarylsulfonylamino, heteroarylhydrocarbylsulfonylamino, cyclohydrocarbylsulfonylamino, heterocyclohydrocarbylsulfonylamino, N-monosubstituted or N,N-disubstituted aminohydrocarbyl group,

wherein the substituent(s) on the aminohydrocarbyl nitrogen atom are selected from the group consisting of hydrocarbyl, aryl, arylhydrocarbyl, cyclohydrocarbyl, arylhydrocarbyloxycarbonyl, hydrocarbyloxycarbonyl, and hydrocarboyl, or wherein the aminohydrocarbyl nitrogen and two substituents attached thereto form a 5- to 8-membered heterocyclic or heteroaryl ring group, arnino, and a N-monosubstituted or N,N-disubstituted amino group,

wherein the substituent(s) on the amino nitrogen are selected from the group consisting of hydrido, hydrocarbyl, aryl, arylhydrocarbyl, cyclohydrocarbyl, arylhydrocarbyloxycarbonyl, hydrocarbyloxycarbonyl, hydrocarboyl, arylsulfonyl, and hydrocarbylsulfonyl or wherein the amino nitrogen and two substituents attached thereto form a 5- to 8-membered heterocyclic or heteroaryl ring group;

Z is selected from the group consisting of hydrido, hydrocarbyl, halogen, carboxy, cyano, azido, hydrocarbylsulfonyl, carbonyloxyhydrocarbyl, carbonylamido, and —X—Y wherein

—X is —O, —S or —NQ,

—Y is hydrido, hydrocarbyl or hydrocarbylaryl,

Q is hydrido, hydrocarbyl, hydroxylhydrocarbyl, 2-, 3-, or 4-pyridylhydrocarbyl, or arylhydrocarbyl;

R¹ is selected from the group consisting of an azido, hydrido, hydrocarbyl, amido, hydrocarbylamino, halohydrocarbyl, perhalohydrocarbyl and an aryl substituent that is optionally substituted by one or more substituents selected from the group consisting of a halogen, hydrocarbyl, hydrocarbyloxy, nitro, cyano, perfluorohydrocarbyl, trifluoromethylhydrocarbyl, hydroxy, mercapto, hydroxycarbonyl, aryloxy, arylthio, arylamino, arylhydrocarbyl, aryl, heteroaryloxy, heteroarylthio, heteroarylamino, heteroarylhydrocarbyl, hydrocarbyloxycarbonylhydrocarbyl, heterocyclooxy, hydroxycarbonylhydrocarbyl, heterocyclothio, heterocycloamino, cyclohydrocarbyloxy, cyclohydrocarbylthio, cyclohydrocarbylamino, heteroarylhydrocarbyloxy, heteroarylhydrocarbylthio, heteroarylhydrocarbylamino, arylhydrocarbyloxy, arylhydrocarbylthio, arylhydrocarbylamino, heterocyclic, heteroaryl, hydroxycarbonylhydrocarbyloxy, alkoxycarbonylalkoxy, hydrocarbyloyl, arylcarbonyl, arylhydrocarbyloyl, hydrocarboyloxy, arylhydrocarboyloxy, hydroxyhydrocarbyl, hydroxyhydrocarbyloxy, hydrocarbylthio, hydrocarbyloxyhydrocarbylthio, hydrocarbyloxycarbonyl, hydroxycarbonylhydrocarbyloxy, hydrocarbyloxy-carbonylhydrocarbyl, hydrocarbylhydroxycarbonyl-hydrocarbylthio, hydrocarbyloxycarbonylhydrocarbyloxy, hydrocarbyloxycarbonylhydrocarbylthio, amino, hydrocarbylcarbonylamino, arylcarbonylamino, cyclohydrocarbylcarbonylamino, heterocyclohydrocarbylcarbonylamino, arylhydrocarbylcarbonylamino, heteroarylcarbonylamino, heteroarylhydrocarbylcarbonylamino, heterocyclohydrocarbyloxy, hydrocarbylsulfonylamino, arylsulfonylamino, arylhydrocarbylsulfonylamino, heteroarylsulfonylamino, heteroarylhydrocarbylsulfonylamino, cyclohydrocarbylsulfonylamino, heterocyclohydrocarbylsulfonylamino and N-monosubstituted or N,N-disubstituted aminohydrocarbyl group,

wherein the substituent(s) on the amino-hydrocarbyl nitrogen atom are selected from the group consisting of hydrocarbyl, aryl, arylhydrocarbyl, cyclohydrocarbyl, arylhydrocarbyloxycarbonyl, hydrocarbyloxycarbonyl, and hydrocarboyl, or wherein the aminohydrocarbyl nitrogen and two substituents attached thereto form a 5- to 8-membered heterocyclic or heteroaryl ring group; and

R² is selected from the group consisting of an azido, hydrido, hydrocarbyl, amido, halohydrocarbyl, perhalohydrocarbyl, hydrocarbyloxycarbonyl, N-piperazinylcarbonyl, aminocarbonyl, piperazinyl and an aryl group that is substituted by one or more substituents, said one or more substituents being selected from the group consisting of a halogen, hydrocarbyl, hydrocarbyloxy, nitro, cyano, perfluorohydrocarbyl, trifluoromethylhydrocarbyl, hydroxy, mercapto, hydroxycarbonyl, aryloxy, arylthio, arylamino, arylhydrocarbyl, aryl, heteroaryloxy, heteroarylthio, heteroarylamino, heteroarylhydrocarbyl, hydrocarbyloxycarbonylhydrocarbyl, heterocyclooxy, hydroxycarbonylhydrocarbyl, heterocyclothio, heterocycloamino, cyclohydrocarbyloxy, cyclohydrocarbylthio, cyclohydrocarbylamino, heteroarylhydrocarbyloxy, heteroarylhydrocarbylthio, heteroarylhydrocarbyamino, arylhydrocarbyloxy, arylhydrocarbylthio, arylhydrocarbylamino, heterocyclic, heteroaryl, hydroxycarbonyl-hydrocarbyloxy, alkoxycarbonylalkoxy, hydrocarbyloyl, arylcarbonyl, arylhydrocarbyloyl, hydrocarboyloxy, arylhydrocarboyloxy, hydroxyhydrocarbyl, hydroxyhydrocarbyloxy, hydrocarbylthio, hydrocarbyloxyhydrocarbylthio, hydrocarbyloxycarbonyl, hydroxycarbonyl-hydrocarbyloxy, hydrocarbyloxycarbonylhydrocarbyl, hydrocarbylhydroxycarbonyl-hydrocarbylthio, hydrocarbyloxycarbonylhydrocarbyloxy, hydrocarbyloxycarbonylhydrocarbylthio, amino, hydrocarbylcarbonylamino, arylcarbonylamino, cyclohydrocarbylcarbonylamino, heterocyclohydrocarbylcarbonylamino, arylhydrocarbylcarbonylamino, heteroarylcarbonylamino, heteroarylhydrocarbylcarbonylamino, heterocyclohydrocarbyloxy, hydrocarbylsulfonylamino, arylsulfonylamino, arylhydrocarbylsulfonylamino, heteroarylsulfonylamino, heteroarylhydrocarbylsulfonylamino, cyclohydrocarbylsulfonylamino, heterocyclohydrocarbylsulfonylamino and N-monosubstituted or N,N-disubstituted aminohydrocarbyl group,

wherein the substituent(s) on the aminohydrocarbyl nitrogen are selected from the group consisting of hydrocarbyl, aryl, arylhydrocarbyl, cyclohydrocarbyl, arylhydrocarbyloxycarbonyl, hydrocarbyloxycarbonyl, and hydrocarboyl, or wherein the aminohydrocarbyl nitrogen and two substituents attached thereto form a 5- to 8-membered heterocyclic or heteroaryl ring group; and

provided that when A is ═CH— and Z is hydrido, hydrocarbyl, halogen, or hydrocarboyl:

1) Ar¹ is other than an aryl group that is substituted by one or more substituents selected from the group consisting of hydrido, halogen, hydrocarbyl, perfluorohydrocarbyloxy, nitro, perfluorohydrocarbyl, amino, aminosulfonyl, halohydrocarbyloxyhydrocarbyl, hydroxy, hydrocarbylsulfonylamino, hydrocarbylsulfonly, acetylamino, carbonylhydrocarbylamino, perfluorohydrocarbylsulfonyl, hydrocarbylamino, carbonyl monosubstituted amino, carbonyl, hydrocarbylthio, hydroxyhydrocarbyl, arylhydrocarbyl, hydrocarbyloxyhydrocarbyl, hydrocarbyloxycarbonyl, hydrocarbyloxyarylhydrocarbyl, halohydrocarbyloxy, hydrocarbyloxyhydrocarbyl; or

2) R¹ is other than hydrido, hydrocarbyl, aryl, haloaryl, cyanoaryl, hydroxyaryl, hydrocarbylaryl, cyano, perfluorohydrocarbyl, hydroxyhydrocarbyl, arylhydrocarbyl, carboxy, hydrocarbyloxycarbonyl, hydrocarboylhydrocarbyl, aminocarbonyl, arylhydrocarbyl-hydrocarboyl-hydrocarbyl monosubstituted amino carbonyl, hydrocarbyl-hydrocarboyl-hydrocarbyl monosubstituted amino carbonyl, hydrocarbyl-hydrocarbylhydrocarboyl-hydrocarbyl monosubstituted amino carbonyl, hydrocarbyl-hydroxy-disubstituted amino carbonylhydrocarbyl, or a six membered heteroaryl group substituted by a nitrogen atom; or

3) R² is other than hydrido, carboxy, hydrocarbyloxycarbonyl, halogen, or aryl.

### Example 12

In some embodiments, the compounds have the structure (Structure 11) described in U.S. Application No. 20020198214 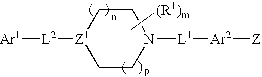
and the pharmaceutically acceptable salts thereof, or a pharmaceutical composition thereof, wherein:

Ar¹ is an aryl group substituted with 0-5 non-interfering substituents, wherein two adjacent noninterfering substituents can form a fused aromatic or nonaromatic ring;

L¹ and L² are linkers;

each R¹ is independently a noninterfering substituent;

Z¹ is CR² or N wherein R² is hydrogen or a noninterfering substituent; mis 0-4;

each of n and p is an integer from 0-2 wherein the sum of n and p is 0-3;

Ar² is a substantially planar, monocyclic or polycyclic aromatic moiety having one or more optional ring heteroatoms, said moiety being optionally substituted with one or more non-interfering substituents, two or more of which may form a fused ring;

Z is —W¹—COXⱼY wherein Y is COR³ or an isostere thereof; R³ is a noninterfering substituent, each of W and X is a spacer of 2-6 angstroms, and each of i and j is independently 0 or 1;

wherein the smallest number of covalent bonds in the compound separating the atom of Ar¹ bonded to L² to the atom of Ar² bonded to L¹ is at least 6, where each of said bonds has a bond length of 1.2 to 2.0 angstroms; and/or wherein the distance in space between the atom of Ar¹ bonded to L² and the atom of Ar² bonded to L¹ is 4.5-24 angstroms;

with the proviso that the portion of the compound represented by Ar²-Z is not

wherein represents a single or double bond; n is 0-3; one Z² is CA or CRA and the other is CR, CR², NR or N; A is —Wᵢ—COXⱼY wherein Y is COR or an isostere thereof, each of W and X is a spacer of 2-6 angstroms, and each of i and j is independently 0 or 1; Z³ is NR or O; and each R is independently hydrogen or a noninterfering substituent.

1-50. (canceled)

51. A method of treating an individual who has been identified as having been infected with HIV and/or who is suspected of having been exposed to HIV who comprising the step of administering to said individual an amount of a p38 inhibitor effective to inhibit FasL expression and/or an amount of a p38 inhibitor effective to inhibit HIV replication without inhibiting T cell activation.

52. The method of claim 51 wherein said p38 inhibitor is administered in an amount effective to inhibit HIV replication.

53. The method of claim 51 wherein said p38 inhibitor is administered in an amount effective to inhibit HIV replication without inhibiting T cell activation.

54. The method of claim 51 wherein said p38 inhibitor is effective at a concentration of less than 1 mM to inhibit FasL expression by at least 50% in greater than 50% of cells in an in vitro assay to measure FasL expression.

55. The method of claim 51 wherein said p38 inhibitor is effective at a concentration of less than 0.1 mM to inhibit FasL expression by at least 50% in greater than 50% of cells in an in vitro assay to measure FasL expression.

56. The method of claim 51 wherein said p38 inhibitor is effective at a concentration of less than 0.05 mM to inhibit FasL expression by at least 50% in greater than 50% of cells in an in vitro assay to measure FasL expression.

57. The method of claim 51 wherein said p38 inhibitor is effective at a concentration of less than 0.01 mM to inhibit FasL expression by at least 50% in greater than 50% of cells in an in vitro assay to measure FasL expression.

58. The method of claim 51 wherein said p38 inhibitor is 4-[5-(4-Fluoro-phenyl)-2-(4-methanesulfinyl-phenyl)-3H-imidazol-4-yl]-pyridine.

59. The method of claim 51 further comprising the step of administering to said individual, in conjunction with said p38 inhibitor, one or more other anti-HIV compounds.

60. The method of claim 51 further comprising the step of administering to said individual, in conjunction with said p38 inhibitor, one or more other anti-HIV compounds selected from the group consisting of: fusion inhibitors (i.e. enfuvirtide), nonnucleoside reverse transcriptase inhibitors (NNRTIs, i.e. delavirdine, efavirenz, nevirapine), nucleoside/nucleotide reverse transcriptase inhibitors (NRTIs, i.e. abacavir, combination of abacavir, lamivudine, and zidovudine, didanosine, lamivudine, combination of lamivudine and zidovudine, stavudine, tenofovir DF, zalcitabine, zidovudine), and protease inhibitors (i.e. amprenavir, indinavir, combination of lopinavir and ritonavir, nelfinavir, ritonavir, saquinavir, invirase).

61. A method of inhibiting FasL expression and/or HIV replication in an HIV infected cell comprising the step of delivering to said HIV infected cell an amount of a p38 inhibitor sufficient to inhibit FasL expression in said cell and/or an amount of a p38 inhibitor sufficient to inhibit HIV replication in said cell, wherein said p38 inhibitor is a p38 inhibitor that does not inhibit T cell activation.

62. The method of claim 61 wherein said p38 inhibitor is delivered in an amount sufficient to inhibit HIV replication.

63. The method of claim 61 wherein said p38 inhibitor is a p38 inhibitor that does not inhibit T cell activation.

64. The method of claim 61 wherein said p38 inhibitor is 4-[5-(4-Fluoro-phenyl)-2-(4-methanesulfinyl-phenyl)-3H-imidazol-4-yl]-pyridine.

65. A method of identifying a compound that inhibits Nef mediated upregulation of FasL expression and screening compounds for inhibition of p38 pathway comprising performing a test assay that comprises the steps of:
a) contacting a cell that expresses Nef with a test compound, wherein said Nef upregulates FasL expression in said cell in the absence of said test compound;
b) measuring the level of FasL expression; and
c) comparing said level with the level of FasL expression in said cells in the absence of said test compound;
wherein a reduction in FasL expression in the presence of said test compound indicates inhibition Nef mediated upregulation of FasL expression and inhibition of the p38 pathway.

66. The method of claim 65 further comprising a negative control assay that comprises the steps of:
a) contacting a cell that expresses Nef with a composition free of any material that effects Nef upregulation of FasL expression, wherein FasL expression in said cell that express Nef is less than FasL expression in said cell prior to expression of Nef;
b) measuring the level of FasL expression; and
c) comparing said level of FasL expression in said negative control assay to the level of FasL expression in said test assay;
wherein a higher level of FasL expression in the negative control assay relative to the test assay indicates that the test compound inhibits Nef mediated upregulation of FasL expression and that the test compound inhibits the p38 pathway.

67. The method of claim 65 further comprising a positive control assay that comprises the steps of:
a) contacting a cell that expresses Nef with a compound that is known to inhibit Nef upregulation of FasL expression, wherein FasL expression in said cell that express Nef is less than FasL expression in said cell prior to expression of Nef;
b) measuring the level of FasL expression; and
c) comparing said level of FasL expression in said positive control assay to the level of FasL expression in said test assay;
wherein the level of FasL expression in the test assay comparable to the level of FasL expression in the positive control assay indicates that the test compound inhibits Nef mediated upregulation of FasL expression and that the test compound inhibits the p38 pathway.

68. The method of claim 65 wherein compounds identified as inhibitors of FasL expression are further screened in a p38 inhibition assay to determine if said compound inhibits p38 activity.

69. The method of claims 65 further comprising the step of performing a different test assay with the test compound to determine p38 inhibitory activity.
